(19) United States
(12) Patent Application Publication
(10) Pub. No.: US 2025/0327033 A1
(43) Pub. Date: Oct. 23, 2025

(54) Methods of Expanding Cholangiocytes

(71) Applicant: Cambridge Enterprise Limited, Cambridge, Cambridgeshire (GB)

(72) Inventors: Fotios Sampaziotis, Cambridge, Cambridgeshire (GB); Ludovic Vallier, Cambridge, Cambridgeshire (GB); Kourosh Saeb-Parsy, Cambridge, Cambridgeshire (GB); Teresa Brevini, Cambridge, Cambridgeshire (GB)

(21) Appl. No.: 18/546,761

(22) PCT Filed: Feb. 16, 2022
(86) PCT No.: PCT/EP2022/053842

§ 371 Date: Aug. 16, 2023

(30) Foreign Application Priority Data
Feb. 17, 2021 (GB) 2102232.2

(51) Int. Cl. C12N 5/071; A61K 35/407; A61K 45/06; A61P 1/16; C12N 5/00; G01N 33/50

(52) U.S. Cl. CPC C12N 5/0679; A61K 35/407; A61K 45/06; A61P 1/16; C12N 5/0062; G01N 33/5067; C12N 2501/11; C12N 2501/415; C12N 2513/00; G01N 2500/10

(57) ABSTRACT

The present invention relates to methods for the expansion of cholangiocytes in vitro that comprise culturing the cholangiocytes in the presence of a farnesoid X receptor (FXR) agonist, such as chenodeoxylic acid (CDA) or obeticholic acid (OCA). The FXR-treated cholangiocytes and organoids obtained by the methods may be useful for example for the treatment of biliary disorders and compound screening. Also provided are kits and uses of culture media for the production of FXR-treated cholangiocyte organoids.

### FUNDING

This work was supported by the European Association for the Study of the Liver (EASL). The project leading to this application has received funding from the European Research Council (ERC) under the European Union's Horizon 2020 research and innovation programme (grant agreement No 741707).

### FIELD

This invention relates to the isolation and propagation of human cholangiocyte organoids, for example for use in disease modelling, drug screening and regenerative medicine.

### BACKGROUND

Organoids have a unique potential for tissue repair as they retain key functions and characteristics of their tissue of origin. Nevertheless, their ability to repair native epithelia and restore their complexity has not been established in humans, while organoid engraftment and survival in vivo has only been demonstrated in a limited number of animal studies (1). The bile duct epithelium presents an archetypal and clinically important system for addressing this challenge and for developing proof-of-concept studies in human. Indeed, disorders of the biliary system, which transfers bile from the liver to the duodenum, account for 70% of paediatric and up to a third of adult liver transplantation (2). This results in a pressing need for therapeutic alternatives, such as cell-based therapy. Furthermore, organoids suitable for regenerative medicine applications can be easily derived from biliary epithelial cells, known as cholangiocytes (3). Finally, the bile ducts also recapitulate the epithelial diversity found in other hollow-lumen organs (4). Indeed, different regions along the biliary tree display distinct transcriptional profiles and functional properties, such as the chemical modification of bile (5, 6), as well as variation in disease susceptibility between the intrahepatic ducts, extrahepatic ducts and the gallbladder.

Nevertheless, the impact of this regional variation on the characteristics and regenerative potential of the organoids derived from different regions of the biliary tree remains to be characterized.

### SUMMARY

The present inventors have recognised that cholangiocyte organoids that are generated in the presence of a farnesoid X receptor (FXR) agonist display a transcriptional profile closer to in vivo cholangiocytes and have improved functionality and regeneration potential relative to previous cholangiocyte organoids. Cholangiocyte organoids generated in this manner may be useful in therapeutic and other applications, for example in regenerative medicine, drug screening and disease modelling.

An aspect of the invention provides a method for expanding cholangiocytes in vitro comprising:
(i) providing a population of cholangiocytes and;
(ii) culturing the population in the presence of a farnesoid X receptor (FXR) agonist to produce an expanded population of FXR treated cholangiocytes (FTCs).

The FXR treated cholangiocytes in the expanded population may be in the form of organoids.

In some preferred embodiments, the population is cultured in an expansion medium comprising the farnesoid X receptor (FXR) agonist, an epidermal growth factor (EGF), a canonical Wnt signalling inhibitor and a non-canonical Wnt signalling potentiator, to produce the expanded population of FXR treated cholangiocytes.

The farnesoid X receptor (FXR) agonist may be a bile acid, such as chenodeoxylic acid (CDA), obeticholic acid (OCA/INT-747), cholic acid (CA), deoxycholic acid (DCA), litocholic acid (LCA), GW4064, Px-102/104, Cilofexor, Tropifexor, Nidufexor and Fexaramine.

The non-canonical Wnt signalling potentiator may be a potentiator of canonical and non-canonical Wnt signalling, preferably R-spondin1.

The canonical Wnt signalling inhibitor may be Dickkopf-related protein 1 (DKK-1).

Preferably, the cholangiocytes are cultured in three-dimensional culture in the expansion medium.

In some embodiments, the method may further comprise disrupting the organoids to produce a population of isolated FXR treated cholangiocytes. The isolated FXR treated cholangiocytes may be further cultured in the expansion medium to expand or propagate the population.

Another aspect of the invention provides a population of isolated FXR treated cholangiocytes produced by a method described herein. The FXR treated cholangiocytes may be in the form of organoids, sub-organoid assemblies or individual cells.

Another aspect of the invention provides a scaffold comprising FXR treated cholangiocytes produced by a method described herein.

Another aspect of the invention provides a method of treatment of a biliary disorder comprising administering a population of isolated FXR treated cholangiocytes or FXR treated cholangiocyte organoids produced as described herein to an individual in need thereof.

Another aspect of the invention provides a method of treatment of a biliary disorder comprising;
administering a population of isolated cholangiocytes or cholangiocyte organoids to an individual in need thereof; and
administering an FXR agonist to the individual.

Preferably, the isolated cholangiocytes or organoids are FXR treated cholangiocytes or organoids.

Another aspect of the invention provides a method of treatment of a biliary disorder comprising;
exposing isolated liver tissue obtained from an individual to an FXR agonist ex vivo; and
transplanting the liver tissue to the individual.

The method may further comprise administering an FXR agonist to the individual.

Another aspect of the invention provides a method of preparing liver tissue for transplant comprising exposing isolated liver tissue to an FXR agonist ex vivo.

Another aspect of the invention provides a method of screening a compound comprising;
contacting a population of the FXR treated cholangiocytes produced as described herein with a test compound, and;
determining the effect of the test compound on the FXR treated cholangiocytes and/or the effect of the FXR treated cholangiocytes on the test compound.

Preferably, the FXR agonist treated cholangiocytes are contacted with the test compound are in the form of organoids (COs).

Another aspect of the invention provides a kit for production of FXR treated cholangiocytes comprising an expansion medium comprising an FXR agonist, epidermal growth factor (EGF), a canonical Wnt signalling inhibitor and a non-canonical Wnt/PCP signalling potentiator.

Another aspect of the invention provides a method for in vitro modelling of a biliary disorder comprising;
(i) providing a population of cholangiocytes from an individual with a biliary disorder and;
(ii) culturing the population in an expansion medium comprising epidermal growth factor (EGF), a canonical Wnt signalling inhibitor and a non-canonical Wnt signalling potentiator, to produce an expanded population of FXR treated cholangiocytes displaying a biliary disorder genotype or phenotype.

Another aspect of the invention provides a method of testing an individual for a biliary disorder comprising;
providing a population of isolated primary cholangiocytes from the individual, expanding the population of cholangiocytes using a method of an aspect of the invention set out above to produce an expanded population of FXR treated cholangiocytes; and
determining the phenotype of the FXR treated cholangiocytes.

Aspects and embodiments of the invention are described in more detail below.

### BRIEF DESCRIPTION OF FIGURES

FIG. 1 shows that cholangiocyte organoid (CO) identity is controlled by niche stimuli and FXR agonists in the niche (in vivo; e.g. bile) or in vitro (CDA) induce a gallbladder identity and shift the transcriptional profile of organoids closer to their in vivo counterparts (A) UMAP (35,603 cells) of primary cholangiocytes and their corresponding organoids before and after gallbladder bile treatment, illustrating similarities between different region organoids and changes in their signature in response to bile. PRI, Primary; IHD, IntraHepatic Ducts; CBD, Common Bile Duct; GB, Gallbladder; ORG, Organoids; BTO, Bile-Treated Organoids. (B) Heatmap of top 100 Differentially Expressed Genes (DEGs) between primary regions, organoids and BTOs (Data S1-S2), illustrating that organoids lose regional differences and upregulate culture-related genes, but re-acquire gallbladder markers following bile treatment. (C-D) QPCR (C) (n=4 samples per group; center line, median; box, interquartile range (IQR); 11 whiskers, range; housekeeping gene, HMBS; #P>0.05, **P<0.01, ***P<0.001, ****P<0.0001); and immunofluorescence (D) demonstrating upregulation of gallbladder markers and bile acid target genes following treatment with chenodeoxycholic acid (CDA), in the absence of the FXR 2 inhibitor Z-GS. Z-GS, Z-guggulsterone. Scale bars, 50 μm.

FIG. 2 shows that cholangiocyte organoids (COs) generated without an FXR agonist rescue cholangiopathy following transplantation and assume an identity corresponding to the site of engraftment. (A) Experimental outline schematic. (B) Kaplan-Meier curve (number of animals at risk) demonstrating animal rescue following gallbladder organoids injection; P=0.0018 (**), log-rank test. (C) Magnetic Resonance Cholangiopancreatography (MRCP) demonstrating rescue of cholangiopathy following organoid injection. (D) Immunofluorescence demonstrating engraftment of Red Fluorescent Protein (RFP)-expressing gallbladder organoids in portal triads, with upregulation of intrahepatic (SOX4) markers. Scale bars; yellow, 50 μm; white, 5 100 μm. PV, portal vein.

FIG. 3 shows that cholangiocyte organoids (COs) generated without an FXR agonist engraft in a human liver receiving Normothermic Perfusion (NMP) and improve bile properties. (A) Schematic representation of the technique for organoid injection and (B) photograph of the NMP circuit used. BD, Bile Duct; GB, Gallbladder; HA, Hepatic Artery; PV, Portal Vein; IVC, Inferior Vena Cava; L, Liver RFP, Red Fluorescent Protein; P, pump; O, oxygenator; PRC, Packed Red Cells. (C) Flow cytometry revealing absence of RFP cells in the perfusate. (D) Immunofluorescence revealing engraftment of RFP gallbladder organoids with upregulation of intrahepatic (SOX4) and loss of gallbladder (SOX17) markers. Scale bars, 50 ηm. (E) Organoid injection improves bile pH and choleresis. ***P<0.001. N=3 NMP livers. Each measurement is represented by a different 10 data point, each organ is represented by a different symbol.

### DETAILED DESCRIPTION

This invention relates to the in vitro expansion of cholangiocytes using a cell culture medium (termed “expansion medium”) comprising an FXR agonist to generate cholangiocytes with phenotype closer to in vivo cholangiocytes (“FXR treated cholangiocytes”).

These FXR treated cholangiocytes may display increased bile resistance in vitro; increased engraftment efficiency; and increased ability to regenerate bile ducts. Furthermore, FXR treated cholangiocytes may display improved functionality and a phenotype closer to in vivo cholangiocytes, and so may be useful for drug screening and disease modelling.

Cholangiocytes are cells from the epithelium of biliary tissue, which is a monolayer covering the luminal surface of the biliary tree. Cholangiocytes play important roles in bile secretion and electrolyte transport in vivo.

Expansion of cholangiocytes in the presence of an FXR agonist as described herein generates an expanded population of cholangiocytes that display a transcriptional profile that resembles the transcriptional profile of primary cholangiocytes in vivo, such as primary gallbladder cholangiocytes. For example, the cholangiocytes may express more markers of primary cholangiocytes, such as FGF19 and SOX17, and expression may be at similar levels to primary cholangiocytes. The cholangiocytes in this expanded population may be referred to as “FXR treated cholangiocytes” or “FTCs” herein.

The transcriptional profile of a cholangiocyte may be determined for example by bulk or scRNAseq or qPCR, followed by data analysis, such as Gene Set Enrichment Analyses (GSEA) or Partition-based Graph Abstraction (PAGA) analysis, as described herein. Marker expression may also be determined by immunofluorescence or Western blot analysis.

FXR treated cholangiocytes and organoids may also demonstrate increase function relative to other expanded populations of cholangiocytes and organoids. For example, FXR treated cholangiocytes and organoids may display higher bile resistance, higher CTFR activity, increased regeneration activity, more efficient bile alkalisation and improved barrier function.

Cholangiocyte function may be determined by standard functional assays, such as CFTR assays, SCR/SST assays, Rhodamine assays and CLF assays (see for example F. Sampaziotis, et al. Nat. Protoc. 12, 814-827 (2017) and WO2018/234323).

Suitable cholangiocytes include primary cholangiocytes, cholangiocytes produced or expanded from primary cholangiocytes using methods available in the art (see for example WO2018/234323) or cholangiocytes produced by in vitro differentiation from pluripotent cells using methods available in the art (see for example Sampaziotis et al Nat Biotech 33 (8) 845-853 (2015), WO2016/207621).

Primary cholangiocytes are isolated directly from the epithelium of intra-or extrahepatic biliary tissue, such as the bile duct or gall bladder and are distinct from continuous (artificially immortalized) biliary cell lines. Primary cholangiocytes may be intra-or extrahepatic cholangiocytes.

Primary cholangiocytes for use as described herein are mammalian, preferably human. Primary cholangiocytes may be obtained from adult or paediatric donors.

The population of primary cholangiocytes does not contain stem cells or other pluripotent or multipotent cells. The differentiation capacity of the primary cholangiocytes in the population is limited to their lineage of origin and they are not able to differentiate into cells of other lineages, such as hepatic or pancreatic cells (i.e. the population consists of cholangiocytes and cholangiocyte precursors).

In some embodiments, the primary cholangiocytes may be cancerous cells, which may be useful for example in drug screening.

Primary cholangiocytes may be obtained or isolated from primary bile tissue in the methods described herein or may have previously been obtained from primary bile tissue. Suitable bile tissue may include the gallbladder and bile ducts from any part of the hepatopancreatobiliary (HPB), pancreatobiliary (PB) or biliary system, including the common bile duct (CBD), cystic duct, common hepatic duct, right hepatic duct, left hepatic duct, intrahepatic ducts and pancreatic duct. Primary bile tissue may for example be obtained from liver explants, liver tissue, liver biopsy, bile duct excision, cholecystectomy, pancreatic resections, endoscopy (ERCP, e.g. biopsies, brushings, spyglass endoscopy, EUS biopsies), or bile (e.g. through ERCP or PTC).

In some preferred embodiments, the cholangiocytes are extrahepatic cholangiocytes. Extrahepatic cholangiocytes originate from the biliary epithelium of the extrahepatic biliary tree and may be obtained from extrahepatic bile tissue, such as the gall bladder, cystic bile duct, common bile duct or common hepatic duct.

In other embodiments, the cholangiocytes are intrahepatic cholangiocytes. Intrahepatic cholangiocytes originate from the biliary epithelium of the intrahepatic biliary tree.

The primary bile tissue from which the cholangiocytes are obtained may be in situ in a donor individual or may be a tissue sample previously obtained from a donor individual, for example after an operation or dissection, such as bile duct excision, liver resection or transplantation, pancreatic resection, cholangioscopy or cholecystectomy. Suitable tissue may be stored in preservation solution before use.

Populations of cholangiocytes may be obtained from primary bile tissue by any convenient technique. In some embodiments, peri-operative techniques may be employed, such as mechanical dissociation of the primary bile tissue for example by brushing or scraping, to dislodge a population of primary cholangiocytes. In other embodiments, minimally invasive techniques, such as Endoscopic Retrograde Cholangio-Pancreatography (ERCP) brushing, may be used.

In some embodiments, populations of cholangiocytes may be obtained by the mechanical dissociation of liver biopsies or explant tissues, for example by plating small (e.g. sub-millimetre) sections of tissue in the culture conditions described herein, with or without the addition of factors such as HGF and forskolin. Alternatively, liver tissue, gallbladder and bile duct explants may be dissociated to single primary cells or small clumps using a combination of mechanical dissociation (scrapping/dicing) and enzymatic digestion using a matrix digesting enzyme, such as liberase, collagenase, or hyalouronidase. Single primary cells may be subsequently be labelled with antibodies for biliary markers, such as EPCAM and isolated with immune isolation methods, such as Magnetic or Fluorescent associated Cell Sorting (MACS or FACS).

Isolated single cells may be plated using the 3D culture conditions described herein or processed for single cell RNA sequencing. The data herein shows that the 3D culture conditions described herein selectively expand cholangiocyte organoids. Other liver cell types, such as hepatocytes, are not propagated in these conditions. This may be shown for example, by the downregulation of hepatic markers (FIG. 28).

The primary bile tissue may be derived from heathy individuals or from patients with known pathology to enable disease modelling.

Cholangiocytes derived from an individual with a biliary disorder may be used to generate expanded populations which display a genotype or phenotype associated with a biliary disorder. A method of producing cholangiocytes with a biliary disorder-associated genotype or phenotype may comprise;
providing a population of primary cholangiocytes from an individual with a biliary disorder,
expanding the primary cholangiocytes as described herein,
thereby producing a population of cholangiocytes with a biliary disorder-associated genotype or phenotype.

An expanded population with a biliary disorder-associated phenotype may display one or more features of the biliary disorder. In some embodiments, the one or more features of the biliary disorder may be displayed in response specific conditions or treatments. For example, the cholangiocytes may be co-cultured with one or more other cell types to elicit a biliary disorder-associated phenotype. For example, the cholangiocytes may be co-cultured with immune cells, such as T-cells, to elicit a phenotype associated with an autoimmune biliary disorder, such as Primary Biliary Cirrhosis (PBC).

Once produced, cholangiocytes with the biliary disorder-associated phenotype may be cultured, expanded and maintained, for example for use in screening.

Cholangiocytes with a biliary disorder-associated phenotype may display one or more properties, features or pathologies characteristic of the biliary disorder.

The expansion medium is a cell culture medium that supports the proliferation of cholangiocytes in the form of organoids (cholangiocyte organoids).

The expansion medium is a nutrient medium which comprises a farnesoid X receptor (FXR) agonist.

The farnesoid X receptor (FXR; NR1H4; Gene ID 9971) is a ligand-activated transcription factor that is activated by bile acids and regulates the expression of genes involved in bile acid synthesis and transport. FXR may have the amino acid sequence of database accession number NP_001193906.1 or an isoform thereof and may be encoded by the nucleotide sequence of NM_001206977.2 or an isoform thereof.

Suitable FXR agonists are known in the art and include bile acids, such as chenodeoxycholic acid (CDA), cholic acid, and obeticholic acid (OCA); cafestol; fexaramine; INT-767; AB23A; Curcumin; Silymarin; Hedragonic acid; Dihydro-artemisinin; Altenusin; PX-102; BAR704; GW 4064; and tropoflexor. Other FXR agonists may be identified using assays available in the art (see for example van der Wiel et al Scientific Reports 9, 2193 (2019); Han Int J Mol Sci. 2018 July; 19 (7): 2069), Hiebl et al. Biotech Adv 2018, 36, 1657

The FXR agonist may be present in the expansion medium at a concentration sufficient to activate or agonise FXR. For example, the expansion medium may contain 1 to 100 μM, preferably about 10 μM of an FXR agonist, such as CDA.

Suitable expansion media for the culture of cholangiocytes are well-known in the art.

In some preferred embodiments, the expansion medium may further comprise EGF, a canonical Wnt inhibitor and a non-canonical Wnt potentiator.

A non-canonical Wnt signalling potentiator is a compound that stimulates, promotes or increases the activity of the non-canonical Wnt signalling pathway.

The non-canonical Wnt signalling pathway is a β-catenin-independent pathway involved in tissue polarity and morphogenetic processes in vertebrates (Komiya, Y. & Habas, R. Organogenesis 4, 68-75 (2008); Patel, V. et al. Hum. Mol. Genet. 17, 1578-1590 (2008); Strazzabosco, M. & Somlo, S. Gastroenterology 140, (2011).)

Components of the non-canonical Wnt signalling pathway include Wnt4, Wnt5a, Wnt11, LRP5/6, Dsh, Fz, Daam1, Rho, Rac, Prickle and Strabismus. Suitable methods for determining the activity of the non-canonical Wnt/PCP signalling pathway are well known in the art and include ATF-2-based reporter assays (Ohkawara et al (2011) Dev Dyn 240 (1) 188-194) and Rho-associated protein kinase (ROCK)-based assays.

A non-canonical Wnt signalling potentiator may selectively potentiate non-canonical Wnt signalling or more preferably, may potentiate both the non-canonical Wnt signalling and the canonical Wnt signalling pathway (i.e. a Wnt signalling agonist).

Preferred non-canonical Wnt signalling potentiators include the Wnt signalling agonist R-spondin.

R-spondin is a secreted activator protein with two cysteine-rich, furin-like domains and one thrombospondin type 1 domain that positively regulates Wnt signalling pathways. Preferably, R-spondin is human R-spondin.

R-spondin may include RSPO1 (GeneID 284654 nucleic acid sequence reference NM_001038633.3, amino acid sequence reference NP_001033722.1), RSPO2 (GeneID 340419 nucleic acid sequence reference NM_001282863.1, amino acid sequence reference NP_001269792.1), RSPO3 (GeneID 84870, nucleic acid sequence reference NM_032784.4, amino acid sequence reference NP_116173.2) or RSPO4 (GeneID 343637, nucleic acid sequence reference NM_001029871.3, amino acid sequence reference NP_001025042.2).

R-spondin is readily available from commercial sources (e.g. R&D Systems, Minneapolis, MN). Suitable concentrations of R-spondin for expanding cholangiocytes as described herein may be readily determined using standard techniques. For example, the expansion medium may comprise 50 ng/ml to 5 μg/ml R-spondin, preferably about 500 ng/ml.

A canonical Wnt signalling inhibitor is a compound that inhibits, blocks or reduces the activity of the canonical Wnt signalling pathway.

The canonical Wnt signalling pathway is a β-catenin-dependent pathway involved in the regulation of gene expression (Klaus et al Nat. Rev. Cancer (2008) 8 387-398; Moon et al (2004) Nat. Rev. Genet. 5 691-701; Niehrs et al Nat Rev Mol. Cell Biol. (2012) 13 763-779) Suitable methods for determining the activity of the canonical Wnt signalling pathway are well known in the art and include the TOP-flash assay (Molenaar et al Cell. 1996 Aug. 9; 86 (3): 391-9) and assays for β-catenin.

Suitable canonical Wnt signalling inhibitors include Dickkopf-related proteins 1-4 (DKKs 1-4), Soggy-1/Dkkl1, secreted Frizzled related proteins 1-5 (sFRP1-5), Wnt inhibitory factor-1 (WIF-1), draxin, SOST/sclerostin, IGFBP-4, USAG1 and Notum.

Preferably, the canonical Wnt signalling inhibitor is DKK-1. DKK-1 (GeneID 22943 nucleic acid sequence reference NM_012242.2, amino acid sequence reference NP_036374.1) is a secreted protein with two cysteine rich regions that plays a role in embryogenesis. DKK-1 is readily available from commercial sources (e.g. R&D Systems, Minneapolis, MN). Suitable concentrations of DKK-1 for expanding cholangiocyte organoids as described herein may be readily determined using standard techniques. For example, the expansion medium may comprise 10 ng/ml to 1 μg/ml DKK-1, for example about 100 ng/ml.

Epidermal Growth Factor (EGF; NCBI GeneID: 1950, nucleic acid sequence NM_001178130.1 GI: 296011012; amino acid sequence NP_001171601.1 GI: 296011013) is a protein factor which stimulates cellular growth, proliferation and cellular differentiation by binding to an epidermal growth factor receptor (EGFR). EGF may be produced using routine recombinant techniques or obtained from commercial suppliers (e.g. R&D Systems, Minneapolis, MN; Stemgent Inc, USA). Suitable concentrations of EGF for expanding cholangiocyte organoids as described herein may be readily determined using standard techniques. For example, the expansion medium may comprise 2 to 500 ng/ml EGF, preferably about 20 ng/ml.

The cholangiocytes may be cultured in the expansion medium in two-dimensional or three-dimensional culture. Preferably, the cholangiocytes are cultured in the expansion medium in three-dimensional culture. For three-dimensional culture, the expansion medium further comprises a scaffold matrix which supports the growth and proliferation of cells in 3-dimensions and allows the cholangiocytes to assemble into organoids.

Suitable scaffold matrices are well-known in the art and include hydrogels, such as collagen, collagen/laminin, compressed collagen (e.g. RAFT™, Lonza Biosystems), alginate, agarose, complex protein hydrogels, such as Base Membrane Extracts, and synthetic polymer hydrogels (Gjorevski et al Nature (2016) 539 560-564), such as polyglycolic acid (PGA) hydrogels and crosslinked dextran and PVA hydrogels (e.g. Cellendes Gmbh, Reutlingen DE), inert matrices, such as porous polystyrene, and isolated natural ECM scaffolds (Engitix Ltd, London UK).

Other suitable scaffold matrices may include decellularised biliary tissue, for example decellularised gallbladder, common bile duct or intra-hepatic bile ducts.

The scaffold matrix may be chemically defined, for example a collagen or densified collagen hydrogel, or non-chemically defined, for example a complex protein hydrogel. Preferably, the scaffold matrix in the expansion medium is a complex protein hydrogel. Suitable complex protein hydrogels may comprise extracellular matrix components, such as laminin, collagen IV, enactin and heparin sulphate proteoglycans. Complex protein hydrogels may also include hydrogels of extracellular matrix proteins from Engelbreth-Holm-Swarm (EHS) mouse sarcoma cells. Suitable complex protein hydrogels are available from commercial sources and include Matrigel™ (Corning Life Sciences) or Cultrex™ BME 2 RGF (Amsbio™ Inc). For example, the expansion medium may comprise 66% Matrigel™.

The expansion medium may comprise or consist of a scaffold matrix and a nutrient medium supplemented with (i) EGF, (ii) a canonical Wnt inhibitor, such as DKK-1, (iii) a non-canonical Wnt potentiator, such as R-spondin and (iv) an FXR agonist, such as CDA.

A nutrient medium may comprise a basal medium. Suitable basal media include Iscove's Modified Dulbecco's Medium (IMDM), Ham's F12, Advanced Dulbecco's modified eagle medium (DMEM) or DMEM/F12 (Price et al Focus (2003), 25 3-6), Williams E (Williams, G. M. et al Exp. Cell Research, 89, 139-142 (1974)), and RPMI-1640 (Moore, G. E. and Woods L. K., (1976) Tissue Culture Association Manual. 3, 503-508. In some embodiments, Williams E medium may be preferred for example 33% Williams E medium.

The basal medium may be supplemented with a media supplement and/or one or more additional components, for example transferrin, 1-thioglycerol, lipids, L-glutamine or substitutes, such as L-alanyl-L-glutamine (e.g. Glutamax™), nicotinamide, linoleic acid and selenous acid (e.g. ITS+ premix), dexamethasone, selenium, pyruvate, buffers, such as HEPES, sodium bicarbonate, phospho-L-ascorbic acid trisodium salt, glucose and antibiotics such as penicillin and streptomycin and optionally polyvinyl alcohol; polyvinyl alcohol and insulin; serum albumin; or serum albumin and insulin.

For example, the basal medium may be supplemented with 10 mM nicotinamide, 17 mM sodium bicarbonate, 0.2 mM 2-phospho-L-ascorbic acid trisodium salt, 6.3 mM sodium pyruvate, 14 mM glucose, 20 mM HEPES, 6 μg/ml insulin, human 6 μg/ml transferrin, 6 ng/ml selenous acid, 5 μg/ml linoleic acid, 0.1 uM dexamethasone, 2 mM L-alanyl-L-glutamine, 100 U/ml penicillin, 100 μg/ml streptomycin.

The nutrient medium may be a chemically defined basal nutrient medium. A chemically defined medium is a nutritive solution for culturing cells which contains only specified components, preferably components of known chemical structure. A chemically defined medium is devoid of undefined components or constituents which include undefined components, such as feeder cells, stromal cells, serum, serum albumin and complex extracellular matrices, such as Matrigel™. A chemically defined medium may be humanised. A humanised chemically defined medium is devoid of components or supplements derived or isolated from non-human animals, such as Foetal Bovine Serum (FBS) and Bovine Serum Albumin (BSA), and mouse feeder cells. Conditioned medium includes undefined components from cultured cells and is not chemically defined.

Suitable chemically defined nutrient media are well-known in the art and include William's E medium supplemented with nicotinamide, sodium bicarbonate, 2-phospho-L-ascorbic acid trisodium salt, sodium pyruvate, glucose, HEPES, ITS+ premix (insulin, transferrin, selenous acid, and linoleic acid), dexamethasone, glutamax, penicillin and streptomycin.

The cholangiocytes may be cultured in the expansion medium for multiple passages. For example, the cholangiocytes may be cultured for 10 or more, 20 or more, 30 or more, 40 or more or 50 or more passages. A passage may take 2-8 days, preferably about 5 days.

The cholangiocytes may be passaged by digesting the scaffold matrix, harvesting cholangiocyte organoids by centrifugation and disrupting the organoids into individual cholangiocytes. The cholangiocytes may be re-suspended and cultured as described above in the expansion medium where they reform into organoids.

Suitable techniques for cell culture are well-known in the art (see, for example, Basic Cell Culture Protocols, C. Helgason, Humana Press Inc. U.S. (15 Oct. 2004) ISBN: 1588295451; Human Cell Culture Protocols (Methods in Molecular Medicine S.) Humana Press Inc., U.S. (9 Dec. 2004) ISBN: 1588292223; Culture of Animal Cells: A Manual of Basic Technique, R. Freshney, John Wiley & Sons Inc (2 Aug. 2005) ISBN: 0471453293, Ho WY et al J Immunol Methods. (2006) 310:40-52, Handbook of Stem Cells (ed. R. Lanza) ISBN: 0124366430) Basic Cell Culture Protocols' by J. Pollard and J. M. Walker (1997), ‘Mammalian Cell Culture: Essential Techniques’ by A. Doyle and J. B. Griffiths (1997), ‘Human Embryonic Stem Cells’ by A. Chiu and M. Rao (2003), Stem Cells: From Bench to Bedside' by A. Bongso (2005), Peterson & Loring (2012) Human Stem Cell Manual: A Laboratory Guide Academic Press and ‘Human Embryonic Stem Cell Protocols’ by K. Turksen (2006). Media and ingredients thereof may be obtained from commercial sources (e.g. Gibco, Roche, Sigma, Europa bioproducts, R&D Systems). Standard mammalian cell culture conditions may be employed for the above culture steps, for example 37° C., 21% Oxygen, 5% Carbon Dioxide. Media is preferably changed every two days and cells allowed to settle by gravity

The population of cholangiocytes may be expanded 10⁵ fold or more, 10¹⁰ fold or more, 1015 fold or more, 10²⁰ fold or 10³⁰ fold or more as organoids in the expansion medium as described herein.

The population of primary cholangiocytes proliferates in the expansion medium and assembles into organoids of gallbladder-like cholangiocytes (COs). Cholangiocyte organoids are three-dimensional multicellular assemblies or cysts that comprise a layer of gallbladder-like cholangiocytes linked by tight junctions which surrounds an interior lumen and separates it from the external environment. The FXR treated cholangiocytes may display polarised expression of markers, such as CFTR.

The organoids formed by the FXR treated cholangiocytes in the expansion medium may display the morphology or physical characteristics of in vivo cholangiocytes, such as cholangiocytes from the gall bladder. For example, the FXR treated cholangiocytes may display columnar with increased height to base ratio and organoids with thicker walls relative to cholangiocytes expanded in the absence of FXR.

Organoids may for example comprise cilia. Tight junctions, microvilli, exosomes and/or tubular structures. The morphology and physical characteristics of organoids may be determined by standard microscopic procedures.

The expanded population of gallbladder-like cholangiocytes, whether in the form of organoids or individual cells, may be free or substantially free from other cell types i.e. the population of cholangiocytes may be homogeneous or substantially homogeneous. For example, the population may contain, 80% or more, 90% or more, 95% or more, 98% or more or 99% or more cholangiocytes, following culture in the medium. Preferably, the population of cholangiocytes is sufficiently free of other cell types that no purification is required.

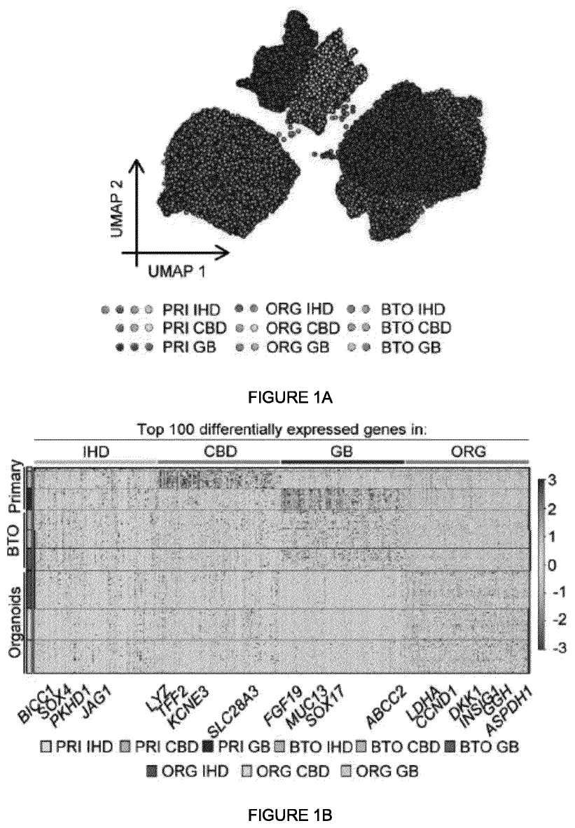

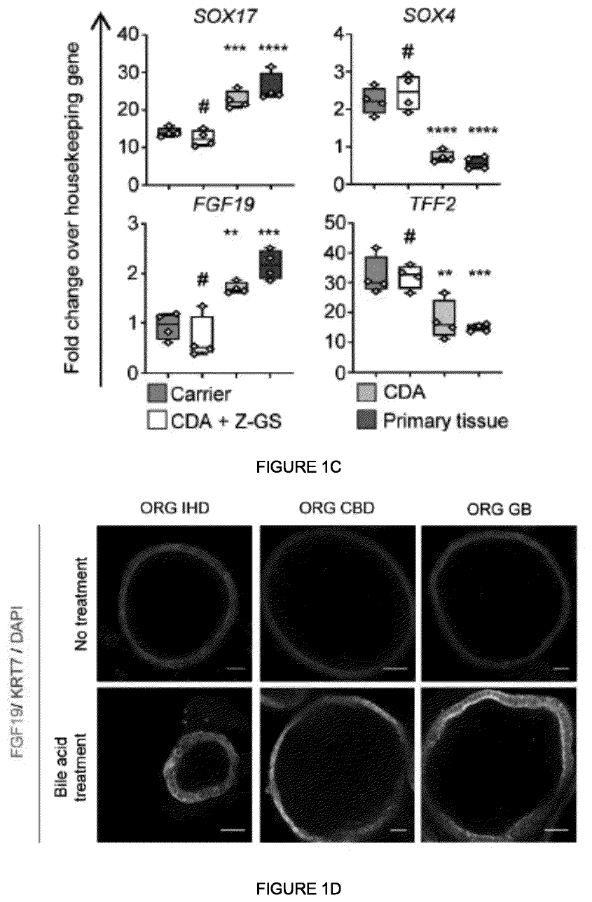

The FXR treated cholangiocytes may express one or more biliary markers. For example, the cholangiocytes may express Cytokeratin 7 (KRT7 or CK7), Cytokeratin 19 (KRT19 or CK19), Gamma Glutamyl-Transferase (GGT), Hepatocyte Nuclear Factor 1 beta (HNF1B), Secretin Receptor (SCTR), Sodium-dependent Bile Acid Transporter 1 (ASBT/SLC10A2), SRY-box 9 (SOX9) Jagged 1 (JAG1), NOTCH2, SCR, SSTR2, Apical Salt and Bile Transporter (ASBT), Aquaporin 1 and Anion Exchanger and Cystic Fibrosis Transmembrane Conductance Regulator (CFTR). Typically, at least 98% of the cholangiocytes in the population may co-express CK7 and CK19 following 20 passages in the expansion medium as described herein. FXR treated cholangiocytes may further express SOX17, FGF19 and other markers shown in the FIG. 1B.

Preferably, the FXR-treated cholangiocytes express mature biliary markers at levels corresponding to primary cholangiocytes. The cholangiocytes may be mature cholangiocytes and may lack foetal characteristics.

In contrast to primary cholangiocytes, the FXR-treated cholangiocytes in the expanded population may lack expression of MHC class 1 or class 2 proteins, for example HLA proteins such as HLA-E or HLA-DRB1. In addition, the cholangiocytes may show expression of genes that are characteristic of primary cholangiocytes of one region of the biliary tree, for example genes induced by bile acid gradient, such as SOX17 and FGF19, and may lack expression of genes characteristic of primary cholangiocytes from a different region of the biliary tree.

In some embodiments, the population of FXR-treated cholangiocytes may be devoid of stem cells or other pluripotent or multipotent cells. For example, the cholangiocytes may display no expression or low expression of stem cell markers, such as POU5F1, OCT4, NANOG, prominin 1 (PROM1), a leucine 4 rich repeat containing G protein-coupled receptor (LGR), such as LGR-4, 5, or 6, Sox2, SSEA-3, SSEA-4, Tra-1-60, KLF-4 and c-myc, relative to control cells. In some preferred embodiments, the cholangiocytes express high levels of biliary markers, low levels of stem cell markers, such as LGR5 and PROM1 and no expression of pluripotency markers, such as Oct4, NANOG and Sox2.

The population of FXR-treated cholangiocytes may be devoid of non-cholangiocyte cells, such as hepatic or pancreatic cells. The cholangiocytes do not express markers of non-biliary lineages, such as hepatocyte or pancreatic markers. For example, the cholangiocytes may lack expression of albumin (ALB), a1-antitrypsin (SERPINA1 or 6 A1AT), pancreatic and duodenal homeobox 1 (PDX1), insulin (INS), glucagon (GCG) and hepatoblast fetal markers, such as AFP.

In some embodiments, the population of FXR-treated cholangiocytes may lack express epithelial-mesenchymal transition (EMT) markers. For example, the cholangiocytes may lack expression of vimentin (VIM), snail family transcriptional repressor 1 (SNAI1) and/or S100 calcium binding protein 9 A4 (S100A4).

The expression of one or more biliary markers and the absence of expression of one or more non-biliary markers may be monitored and/or detected in the expanded population of FXR-treated cholangiocytes. For example, the expression or production of one or more of the mature biliary markers set out above in the expanded population of FXR-treated cholangiocytes may be determined. This allows the homogeneity of the expanded population of FXR-treated cholangiocytes to be determined and/or monitored.

The expanded population of FXR-treated cholangiocytes produced as described herein may display in vitro one or more functional properties of primary cholangiocytes, such as primary gallbladder (GB) cholangiocytes. For example, the cholangiocytes may assemble into organoids that display one or more, preferably all of the properties described below.

The FXR-treated cholangiocyte organoids may display bile acid transfer, alkaline phosphatase (ALP) activity and/or Gamma-Glutamyl-Transpeptidase (GGT) activity. The amount of ALP and GGT activity may correspond to the amount of ALP and GGT activity displayed by primary cholangiocytes. ALP and GGT activity may be determined, for example, as described herein.

The FXR-treated cholangiocyte organoids may display active secretion, for example, secretion mediated by multidrug resistance protein-1 (MDR1). This may be determined by measuring the accumulation of a fluorescent MDR1 substrate, such as Rhodamine123, in the lumen of FXR-treated cholangiocyte organoids in the presence and absence of MDR1 inhibitor verapamil, as described herein.

The FXR-treated cholangiocyte organoids may display responses to secretin and somatostatin. For example, the cholangiocyte organoids may display increased secretory activity in response to secretin and decreased activity in response to somatostatin. This may be determined by measuring changes in organoid size. For example, secretin may increase and somatostatin may decrease the size of cholangiocyte organoids.

The FXR-treated cholangiocyte organoids may display active transfer of bile acids, for example transfer mediated by Apical Salt and Bile Transporter (ASBT). Bile acid transfer activity may be determined, for example, by measuring the active transfer of a fluorescent bile salt, such as CLF, relative to another fluorescent compound, such as FITC, as described herein.

The FXR-treated cholangiocyte organoids may display Cystic Fibrosis Transmembrane Conductance Regulator (CFTR) activity. CTFR activity may be determined by measuring intracellular and intraluminal chloride concentrations in response to media with varying chloride concentrations, for example, the fluorescent chloride indicator N-(6-methoxyquinolyl) acetoethyl ester (MQAE), as described herein.

The FXR-treated cholangiocyte organoids may display responses to ATP and acetylcholine. For example, intracellular Ca²⁺ levels may increase in the cholangiocyte organoids in response to ATP or acetylcholine. Intracellular Ca²⁺ levels may be determined using standard techniques.

The FXR-treated cholangiocyte organoids may display responses to Vascular Endothelial Growth Factor (VEGF), Mitogens such as IL6, and oestrogens. For example, the cholangiocyte organoids may display increased proliferation in response to VEGF.

The cholangiocyte organoids may display responses to drugs, such as lumacaftor (VX809). For example, size, CFTR activity and/or intraluminal fluid secretion may increase in response to lumacaftor in cholangiocyte organoids expanded from primary cholangiocytes obtained from a donor individual with cystic fibrosis. Suitable methods for determining responses to lumacaftor are described below.

The amount of response and/or activity of the FXR-treated cholangiocyte organoids produced by the claimed methods may correspond to the amount of response and/or activity displayed by primary cholangiocytes, preferably primary intrahepatic cholangiocytes or primary extrahepatic cholangiocytes, such as common bile duct (CBD) cholangiocytes. In addition, the amount of response and/or activity of the FXR-treated cholangiocyte organoids produced by the claimed methods may be greater than the amount of response and/or activity of the cholangiocyte organoids generated in the absence of FXR agonists.

Following the expansion in expansion medium, as described above, FXR-treated cholangiocyte organoids may be dissociated or disrupted to generate individual FXR-treated cholangiocytes.

Suitable methods of dissociating organoids into individual constituent cells are well-known in the art (see for example F. Sampaziotis, et al. Nat. Protoc. 12, 814-827 (2017)). For example, the cholangiocyte organoids may be harvested from the expansion medium using a dispase or non-enzymatic recovery solution, such as Cell Recovery Solution™ (Corning) and dissociated using a protease, such as trypsin. Suitable reagents are commercially available and include TrypLE™ Express (ThermoFisher Scientific).

The ability of FXR-treated cholangiocytes expanded as described herein to perform one or more cholangiocyte functions may be monitored and/or determined. For example, the ability of the cells to assemble into organoids, and/or perform one or more of MDR1 function; bile acid transfer; VEGF, acetylcholine or ATP responses; CFTR mediated chloride transport; or secretin or somatostatin responses may be monitored and/or determined.

FXR-treated cholangiocytes produced as described herein may be expanded as described herein or cultured or maintained using standard mammalian cell culture techniques (see for example F. Sampaziotis, et al. Nat. Protoc. 12, 814-827 (2017)) or subjected to further manipulation or processing. In some embodiments, the cholangiocyte populations produced as described herein may be stored, for example by lyophilisation and/or cryopreservation. The FXR-treated cholangiocytes may be stored as organoids, sub-organoid assemblies or individual cells. Suitable storage methods are well known in the art. For example, the cholangiocytes may be suspended in a cryopreservation medium (for example, Cellbanker™ (AMS Biotechnology Ltd, UK) and frozen, for example at −70° C. or below.

The population of FXR-treated cholangiocytes may be admixed with other reagents, such as buffers, carriers, diluents, preservatives, and/or pharmaceutically acceptable excipients. Suitable reagents are described in more detail below. A method described herein may comprise admixing the population of cholangiocytes with a therapeutically acceptable excipient to produce a therapeutic composition. The admixed FXR-treated cholangiocytes may be in the form of organoids, sub-organoid assemblies or individual cells.

In some embodiments, the FXR-treated cholangiocytes produced as described herein display increased regenerative capability than cholangiocytes produced without FXR agonism and may be useful in therapy. For therapeutic applications, the FXR-treated cholangiocytes are preferably clinical grade cells. Populations of FXR-treated cholangiocytes for use in treatment are preferably produced from primary cholangiocytes as described herein using a chemically defined expansion medium. The FXR-treated cholangiocytes may be in the form of organoids, sub-organoid assemblies or individual cells, depending on the specific application.

The expanded population of FXR-treated cholangiocytes may be transplanted, infused or otherwise administered into the individual. Suitable techniques are well known in the art.

The expanded population of FXR-treated cholangiocytes may be autologous i.e. the FXR-treated cholangiocytes were expanded from primary cholangiocytes originally obtained from the same individual to whom they are subsequently administered (i.e. the donor and recipient individual are the same). A suitable expanded population of FXR-treated cholangiocytes for administration to a recipient individual may be produced by a method comprising providing an initial population of primary cholangiocytes obtained from the individual and expanding the population of cholangiocytes as described above to produce an expanded population of FXR-treated cholangiocytes for administration.

The expanded population of FXR-treated cholangiocytes may be allogeneic i.e. the primary cholangiocytes were originally obtained from a different individual to the individual to whom the FXR-treated cholangiocytes are subsequently administered (i.e. the donor and recipient individual are different). The donor and recipient individuals may be HLA matched and/or blood group matched to avoid rejection and other undesirable immune effects. The donor and recipient individuals may also be matched for previous viral infection history and/or viral immunity status e.g. previous CMV infection. In some embodiments, the recipient individual may receive immunosuppression therapy to reduce or prevent rejection of the allogeneic FXR treated cholangiocytes. A suitable expanded population of FXR-treated cholangiocytes for administration to a recipient individual may be produced by a method comprising providing an initial population of primary cholangiocytes obtained from a donor individual, and expanding the population of cholangiocytes as described above to produce an expanded population of FXR-treated cholangiocytes for administration. In some embodiments, the expanded population may be engineered to reduce or inactivate the expression of immunogenic antigens, such as HLAs and/or the recipient individual may be treated with one or more immunosuppressive agents.

In some preferred embodiments, the expanded population of FXR-treated cholangiocytes may be admixed with a biocompatible scaffold, such as decellularized human extracellular matrix or decellularized non-human (e.g. porcine) extracellular matrix.

A biocompatible scaffold may be seeded with cholangiocytes expanded as described above. For example, individual cholangiocytes or sub-organoid assemblies of FXR-treated cholangiocytes may be injected on or into a scaffold or mixing into the scaffold during the manufacturing process. The scaffold containing the cholangiocytes may then be cultured in expansion medium, such that the cholangiocytes populate the scaffold. The cholangiocytes may proliferate within the scaffold and assemble into organoids and then into a multi-layered epithelium.

Suitable biocompatible scaffolds may include hydrogels, such as fibrin, chitosan, glycosaminoglycans, silk, fibrin, fibronectin, elastin, collagen, glycoproteins such as fibronectin, or polysaccharides such as chitin, or cellulose collagen, collagen/laminin, densified collagen, alginate, agarose, complex protein hydrogels, such as Base Membrane Extracts, bio-organic gels, and synthetic polymer hydrogels, such as polylactic acid (PLA) polyglycolic acid (PGA), polycapryolactone (PCL) hydrogels, crosslinked dextran and PVA hydrogels (e.g. Cellendes Gmbh, Reutlingen DE), inert matrices, such as porous polystyrene, polyester, soluble glass fibres porous polystyrene, and isolated natural ECM scaffolds, for example decellularized gall bladder and bile duct scaffolds (Engitix Ltd, London UK). The scaffold may be biodegradable.

The size or shape of the scaffold is dependent on the intended application. Suitable scaffold shapes may for example include patches, sheets and tubes, including straight and branched tubes, with diameters up to for example 10-12 mm.

FXR-treated cholangiocytes produced as described herein that are cultured within a biocompatible scaffold organize into a functional biliary epithelium. The populated scaffold may display one or more properties of the biliary epithelium. For example, the populated scaffold may be bile resistant and may display one or more of the functional properties described above. A scaffold populated with FXR-treated cholangiocytes may be useful as artificial biliary epithelial tissue, for example for use in therapy or screening.

Another aspect of the invention provides a population of isolated FXR-treated cholangiocytes produced by a method described herein. The population may be in the form of organoids, sub-organoid assemblies or clusters or individual cells.

A population of FXR-treated cholangiocytes generated as described herein may be substantially free from other cell types. For example, the population may contain 70% or more, 80% or more, 85% or more, 90% or more, or 95% or more FXR-treated cholangiocytes, following culture in the expansion medium. The presence or proportion of FXR-treated cholangiocytes in the population may be determined through the expression of biliary markers as described above.

Preferably, the population of FXR-treated cholangiocytes is sufficiently free of other cell types that no purification is required. If required, the population of cholangiocytes or cholangiocyte organoids may be purified by any convenient technique, including FACS.

In some embodiments, the FXR-treated cholangiocytes may be engineered to express a heterologous protein, for example a marker protein, such as GFP, or an enzyme and/or to reduce or prevent expression of one or more endogenous protein, for example proteins associated with immunogenicity. For example, the cholangiocytes may be transfected with a vector comprising a nucleic acid encoding a heterologous protein; a suppressor RNA which suppresses the expression of an endogenous protein; or a site specific nuclease that inactivates an endogenous protein. In some embodiments, the cholangiocytes may be engineered to correct a genetic defect. For example, defects in the CFTR gene may be corrected in cholangiocytes derived from an individual with cystic fibrosis. In other embodiments, the cholangiocytes may be engineered to remove immunogenic antigens, such as human leukocyte antigens (HLA). This may be useful in generating low or non-immunogenic cells for allogenic use.

Another aspect of the invention provides a scaffold comprising cholangiocytes by a method described herein. Suitable scaffolds are described above.

Another aspect of the invention provides an artificial biliary epithelium tissue comprising a scaffold populated with FXR-treated cholangiocytes produced by a method described herein, for example for use in therapy. In addition to FXR-treated cholangiocytes, an artificial tissue may incorporate other cells, such as stromal and/or endothelial cells.

Aspects of the invention also extend to a pharmaceutical composition, medicament, drug or other composition comprising cholangiocytes produced as described herein in solution or in a biocompatible scaffold, and a method of making a pharmaceutical composition comprising admixing such cholangiocytes with a pharmaceutically acceptable excipient, vehicle, carrier or biodegradable scaffold, and optionally one or more other ingredients.

A pharmaceutical composition containing FXR-treated cholangiocytes expanded in accordance with the invention may comprise one or more additional components. For example, in addition to the cholangiocytes, a pharmaceutical composition may comprise a pharmaceutically acceptable excipient, carrier, buffer, preservative, stabiliser, anti-oxidant, or other material well known to those skilled in the art. Such materials should be non-toxic and should not interfere with the activity of the cholangiocytes. The precise nature of the carrier or other material will depend on the route of administration.

Liquid pharmaceutical compositions generally include a liquid carrier such as water, petroleum, animal or vegetable oils, mineral oil or synthetic oil. Physiological saline solution, tissue or cell culture media, dextrose or other saccharide solution or glycols such as ethylene glycol, propylene glycol or polyethylene glycol may be included.

The composition may be in the form of a parenterally acceptable aqueous solution, which is pyrogen-free and has suitable pH, isotonicity and stability. Those of relevant skill in the art are well able to prepare suitable solutions using, for example, isotonic vehicles such as Sodium Chloride, Ringer's Injection, or Lactated Ringer's Injection. A composition may be prepared using artificial cerebrospinal fluid.

Another aspect of the invention provides a method of treatment of a biliary disorder or a liver disease comprising administering a population of FXR-treated cholangiocytes produced as described herein to an individual in need thereof.

Another aspect of the invention provides a population of FXR-treated cholangiocytes produced as described herein for use in a method of treatment of a biliary disorder or a liver disease in an individual in need thereof comprising administering a the population to the individual.

Another aspect of the invention provides the use of a population of FXR-treated cholangiocytes produced as described herein in the manufacture of a medicament for use in the treatment of a biliary disorder or a liver disease.

The FXR-treated cholangiocytes may be in the form of organoids, sub-organoid assemblies or clusters or individual cells.

A biliary disorder is a condition in which the biliary tissue in an individual is damaged, defective or otherwise dysfunctional, for example, disorders characterised by damage to or destruction of bile ducts, aberrant bile ducts or the absence of bile ducts. Biliary disorders may include biliary tissue injury, ischaemic strictures, traumatic bile duct injury and cholangiopathies, for example inherited, developmental, autoimmune and environment-induced cholangiopathies, such as Cystic Fibrosis associated cholangiopathy, drug induced cholangiopathy, Alagille Syndrome, polycystic liver disease, primary biliary cirrhosis (PBC), primary sclerosing cholangitis (PSC), AIDS associated cholangiopathy, disappearing bile duct syndrome, biliary cancer, ductopenias such as adult idiopathic ductopenia, post-operative biliary complications, biliary atresia, Xanthogranulomatous cholecystitis (XGC), gallbladder adenomyomatosis, gallbladder infections e.g. Salmonella, gallbladder cancer, and other disorders of the extra-or intrahepatic bile ducts.

In some embodiments, an expanded population of FXR-treated cholangiocytes may be administered to the individual in suspension. The administration of a population of FXR treated cholangiocytes in suspension may be useful for example in the treatment of liver disease and biliary disorders.

Liver disease may include cholangiopathies, for example ductopenias, such as ischaemic ductopenia, congenital ductopenia, such as alagille syndrome, metabolic ductopenia, and drug induced ductopenia; complex diseases, such as intrahepatic primary sclerosing cholangitis (PSC) and primary biliary cholangitis (PBC),, vanishing bile duct syndrome; post-transplant cholangiopathy, and conditions affecting the intrahepatic biliary tree

In other embodiments, a population of FXR-treated cholangiocytes may be administered to the individual within a biocompatible scaffold. Suitable scaffolds may include decellularised organs, such as bile ducts, that have been seeded or repopulated with FXR-treated cholangiocytes. For example, a scaffold populated with cholangiocytes may be administered to the individual. The administration of a population of cholangiocytes in a scaffold may be useful for example in the treatment of biliary atresia, biliary strictures, traumatic or iatrogenic biliary injury and conditions affecting the extrahepatic biliary tree

Cholangiocytes in solution or in scaffolds may be implanted into a patient by any technique known in the art (e.g. Lindvall, O. (1998) Mov. Disord. 13, Suppl. 1:83-7; Freed, C. R., et al., (1997) Cell Transplant, 6, 201-202; Kordower, et al., (1995) New England Journal of Medicine, 332, 1118-1124; Freed, C. R., (1992) New England Journal of Medicine, 327, 1549-1555, Le Blanc et al, Lancet 2004 May 1; 363 (9419): 1439-41). In particular, cell suspensions may be injected or infused into the bile duct, gallbladder, portal vein, liver parenchyma, peritoneal cavity or spleen of a patient. A cholangiocyte suspension may be administered intravenously, intrasplenically, intraperitoneally or via an endoscopic retrograde cholangio-pancreatography (ERCP) or percutaneous cholangiography (PTC). A scaffold populated with cholangiocytes may be administered to the individual by surgical implantation.

Administration of a composition in accordance with the present invention is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc., is within the responsibility of general practitioners and other medical doctors.

A composition comprising FXR-treated cholangiocytes may be administered alone or in combination with other treatments, either simultaneously or sequentially dependent upon the condition to be treated.

Methods of the invention may be useful in the treatment of patients receiving transplants of cholangiocytes, for example for the treatment of a liver disease or biliary disorder. For example, a method of treatment of a biliary disorder may comprise;
administering a population of isolated cholangiocytes or cholangiocyte organoids to an individual in need thereof; and administering an FXR agonist to the individual.
Administration of the FXR agonist may improve the function or regenerative ability of the cholangiocytes or cholangiocyte organoids.

In some embodiments, the cholangiocytes or cholangiocyte organoids may be FXR treated cholangiocytes or cholangiocyte organoids. For example, FXR treated cholangiocytes or cholangiocyte organoids produced as described above. In other embodiments, the cholangiocytes or cholangiocyte organoids may be produced without FXR treatment and may be exposed an FXR agonist after administration to the individual.

Methods of the invention may be useful in the treatment of liver tissue ex vivo. A method of preparing liver tissue for transplant may comprise;
exposing isolated liver tissue to an FXR agonist ex vivo.

Exposure of the liver tissue to the FXR agonist may improve the functional properties and regenerative abilities of the cholangiocytes therein

This may be useful for example in treating a liver disease or biliary disorder. A method of treatment of a biliary disorder may comprise;
exposing isolated liver tissue obtained from an individual to an FXR agonist ex vivo; and
transplanting the liver tissue to the individual following said exposure.

A method may further comprise administering an FXR agonist to the individual following transplantation of the liver tissue to the individual. This may be useful for example in maintaining the cells improved function or regenerative abilities following engraftment.

Other aspects of the invention relate to the use of FXR-treated cholangiocytes expanded as described herein to determine the susceptibility of a patient to a drug. A method may comprise
(i) providing a population of isolated primary cholangiocytes from an individual with a disease condition, such as a biliary disorder or liver disease and;
(ii) culturing the population in an expansion medium comprising an FXR agonist, epidermal growth factor (EGF), a canonical Wnt signalling inhibitor and a non-canonical Wnt/PCP signalling potentiator, to produce an expanded population of FXR-treated cholangiocytes displaying a disease phenotype,
(iii) contacting the expanded population of FXR-treated cholangiocytes produced by a method described herein with a therapeutic compound, and;
(iv) determining the effect of the therapeutic compound on said FXR-treated cholangiocytes,
wherein an amelioration of the disease phenotype of the FXR-treated cholangiocytes is indicative that the individual is susceptible to the therapeutic compound.

The proliferation, growth, viability or bile acid resistance of FXR-treated cholangiocytes, their ability to perform one or more cell or organoid functions as described below or their ability to perform one or more of (i) engraft to a non-human animal model following transplantation (ii) form bile ducts in vivo in a non-human animal model, (iii) rescue the disease phenotype in vivo in a non-human animal model (iv) prolong survival of a non-human animal model after transplantation, (v) maintain cell function in vivo in a non-human animal model, (vi) reverse ductopenia in vivo, (vii) improve serum liver function markers following transplantation in a non-human animal model, and/or (viii) engraft, repair ducts and improve function of human organs maintained ex-vivo, may be determined in the presence relative to the absence of the therapeutic compound.

An increase in the ability of the expanded FXR-treated cholangiocytes with the disease phenotype to perform one or more of these functions in the presence relative to the absence of the therapeutic compound is indicative that the compound has a ameliorative effect on the disease in the individual.

Populations of isolated FXR-treated cholangiocytes produced as described above may be useful in modelling the interaction of test compounds with cholangiocytes, for example in toxicity screening, modelling biliary disorders or screening for compounds with potential therapeutic effects.

In some embodiments, cholangiocytes may be obtained from healthy primary tissue. In other embodiments, cholangiocytes may be obtained from primary tissue from a donor with a biliary disease and may display a disease phenotype.

Another aspect of the invention provides the use of a population of FXR-treated cholangiocytes derived from a normal patient or a patient with a biliary disorder for disease modelling and study of pathogenesis of biliary disorders.

FXR-treated cholangiocytes for use in modelling and screening may be in the form of organoids (cholangiocyte organoids), sub-organoid clusters or individual cells (cholangiocytes) produced, for example by disruption of cholangiocyte organoids.

A method of screening a compound may comprise;
contacting a population of FXR-treated cholangiocytes produced by a method described herein with a test compound, and;
determining the effect of the test compound on said the cholangiocytes and/or the effect of said the cholangiocytes on the test compound.

The proliferation, growth, viability or bile acid resistance of cholangiocytes, or their ability to perform one or more cell or organoid functions may be determined in the presence relative to the absence of the test compound.

A decrease in proliferation, growth, viability or ability to perform one or more cell or organoid functions is indicative that the compound has a toxic effect and an increase in growth, viability or ability to perform one or more cell or organoid functions is indicative that the compound has an ameliorative effect on the cholangiocytes.

In some embodiments, the FXR-treated cholangiocytes may be derived from biliary tumours and the effect of the test compound on the proliferation, growth, viability or ability to perform one or more cell or organoid functions of the tumour derived cells may be determined.

Gene expression may be determined in the presence relative to the absence of the test compound. For example, the expression of one or more biliary marker genes may be determined. Combined decrease in expression is indicative that the compound has a toxic effect or can modify the functional state of the cholangiocytes. Gene expression may be determined at the nucleic acid level, for example by RT-PCR, or at the protein level, for example, by immunological techniques, such as ELISA, or by activity assays. Cytochrome p450 assays, for example, luminescent, fluorescent or chromogenic assays are well known in the art and available from commercial suppliers.

In some embodiments, the expression of risk loci for a biliary disease or genes associated with a biliary disease, for example a disease described above, may be determined.

The metabolism, degradation, or breakdown of the test compound by the FXR-treated cholangiocytes may be determined. In some embodiments, changes in the amount or concentration of test compound and/or a metabolite of said test compound may be determined or measured over time, either continuously or at one or more time points. For example, decreases in the amount or concentration of test compound and/or increases in the amount or concentration of a metabolite of said test compound may be determined or measured. In some embodiments, the rate of change in the amount or concentration of test compound and/or metabolite may be determined. Suitable techniques for measuring the amount of test compound or metabolite include mass spectrometry.

This may be useful in determining the in vivo half-life, toxicity, efficacy or other in vivo properties of the test compound.

One or more functions of the FXR-treated cholangiocytes may be determined and/or measured in the presence relative to the absence of the test compound. For example, the ability of the cholangiocytes to perform one or more of MDR1 function; bile acid transfer; VEGF, acetylcholine or ATP responses; CFTR mediated chloride transport; GGT activity, ALP activity or secretin or somatostatin responses, forskolin-induced swelling (Dekkers et al Nat Med 2013; 19:939-45) bile resistance, bicarbonate secretion, lumen integrity (i.e. does the compound does the compound break tight junctions and collapse the lumen of an organoid), transfer of compound in and out of the organoid lumen and the presence or viability of bacteria in the lumen, may be determined and/or measured. The ability of the cholangiocytes to assemble into cholangiocyte organoids may also be determined.

A decrease in the ability of the FXR-treated cholangiocytes to perform one or more of these functions in the presence relative to the absence of the test compound is indicative that the compound has a toxic effect on the biliary epithelium. An increase in the ability of the cholangiocytes to perform one or more of these functions in the presence relative to the absence of the test compound is indicative that the compound has a pro-biliary effect (e.g. it promotes the activity of the biliary epithelium).

Another aspect of the invention provides a kit for production of FXR-treated cholangiocyte organoids comprising an expansion medium comprising an FXR agonist.

The kit may further comprise epidermal growth factor (EGF), a non-canonical Wnt/PCP signalling potentiator and a canonical Wnt signalling inhibitor.

Suitable expansion media are described in more detail above.

The kit may further comprise a scaffold matrix, such as Matrigel™. The scaffold matrix may be provided as part of the expansion medium or may be provided separately.

The expansion medium may be formulated in deionized, distilled water. The expansion medium will typically be sterilized prior to use to prevent contamination, e.g. by ultraviolet light, heating, irradiation or filtration. The one or more media may be frozen (e.g. at −20° C. or −80° C.) for storage or transport. The one or more media may contain one or more antibiotics to prevent contamination.

The kit may further comprise a sampler, such as a brush or scrapper, for the isolation of primary cholangiocytes from primary bile tissue. The kit may further comprise plates or vessels for mechanical isolation of cholangiocytes from tissue samples and centrifuge tubes for separating cells from tissue debris.

The kit may further comprise a preservation medium to preserve the tissue before the extraction of the cells. Suitable media include UW solution (e.g. Vivaspin™) and William's E medium supplemented with pro-survival cytokines and/or Rock inhibitor.

The kit may further comprise a wash medium. Suitable wash media may include William's E medium supplemented with EGF and Rock inhibitor.

The kit may further comprise pro-survival cytokines such as ROCK-inhibitors

The kit may further comprise a plate heater.

The kit may further comprise cryopreservation solution. Suitable cryopreservation media are described above.

The one or more media may be a 1× formulation or a more concentrated formulation, e.g. a 2× to 250× concentrated medium formulation. In a 1× formulation each ingredient in the medium is at the concentration intended for cell culture, for example a concentration set out above. In a concentrated formulation one or more of the ingredients is present at a higher concentration than intended for cell culture. Concentrated culture media are well known in the art. Culture media can be concentrated using known methods e.g. salt precipitation or selective filtration. A concentrated medium may be diluted for use with water (preferably deionized and distilled) or any appropriate solution, e.g. an aqueous saline solution, an aqueous buffer or a culture medium.

The one or more media in the kit may be contained in hermetically-sealed vessels. Hermetically-sealed vessels may be preferred for transport or storage of the culture media, to prevent contamination. The vessel may be any suitable vessel, such as a flask, a plate, a bottle, a jar, a vial or a bag.

Another aspect of the invention provides a use of an expansion medium for the in vitro expansion of FXR-treated cholangiocytes, wherein the expansion medium comprises an FXR agonist.

The expansion medium may further comprise epidermal growth factor (EGF), a non-canonical Wnt signalling potentiator and a canonical Wnt signalling inhibitor.

Suitable additional components of an expansion medium are known in the art (see for example F. Sampaziotis, et al. Nat. Protoc. 12, 814-827 (2017)).

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Certain aspects and embodiments of the invention will now be illustrated by way of example and with reference to the figures described above.

### Experiments

### 1. Methods

### Tissue Collection

Gallbladder, bile duct, liver biopsies and bile were obtained under sterile conditions from deceased transplant organ donors as rapidly as possible after cessation of circulation. Tissue samples, and liver retrieved for transplantation but subsequently declined, were transferred to the laboratory at 4° C. in University of Wisconsin (UW®) organ presentation solution.

### Tissue Dissociation

Resected tissue (gallbladder, extrahepatic ducts and liver) was transferred to the lab as described above and processed immediately after resection. Gallbladder and extrahepatic bile duct samples were drained of bile and the organ lumen was exposed through a longitudinal incision. Liver samples were divided into 1 cm2 cubes prior to processing. All samples were washed twice with warm PBS with Ca2+Mg2++EDTA (0.5 mM), followed by enzymatic digestion with using Liberase (0.2 Wunsch/ml) in an incubated shaker at 37° C. and 200 RPM for 30 minutes. DNAse I (2000 U/ml) was added to the solution to prevent cell clumping and increase viability. Liver samples were dissociated further using the Miltenyi Biotec GentleMACS tissue dissociator and GentleMacs Tissue Dissociation C Tubes. For the gallbladder and extrahepatic duct samples, gentle mechanical scrapping of the lumen was adequate to release the epithelial cells following enzymatic digestion. All cell suspensions were filtered through 70 um filters to remove debris and remaining tissue, washed with PBS containing 1% BSA (W/V) and centrifuged at 400 g, for 5 mins in a refrigerated centrifuge maintaining a temperature of 4° C. The cells were resuspended in Miltenyi Biotec red blood cell (RBC) lysis and incubated for 10 minutes at room temperature (RT). The Miltenyi Biotec Debris Removal solution kit was used according to the manufacturer's instructions to remove remaining debris and dead cells. For liver samples, the resulting cell suspensions were centrifuged at 50 g for 5 minutes (4° C.) to pellet the hepatocyte fraction, the supernatant was collected and cholangiocytes were isolated as described below

### Cell Isolation

Following tissue dissociation to single cells, cholangiocytes were isolated with Magnetic Associated Cell Sorting (MACS) using the Miltenyi Biotech autoMACS Pro separator and CD326 (EpCAM) MicroBeads according to the manufacturer's instructions. The resulting cells were counted, centrifuged at 444 g for 5 minutes, resuspended to a concentration of 1000 cells/μL and stored on ice.

### 10× Single Cell Library Making Process

GEM-RTs (Gel-beads-in-emulsion which barcode the ploy adenylated mRNAs, followed by Reverse Transcription) were broken and silane magnetic beads are used to purify first strand cDNA from the GEM-RT mixture and the cDNA was then amplified via PCR. Enzymatic fragmentation, end-repair and A-tailing were followed by size selection (using SPRISelect reagent). An adapter was ligated to the fragments and following a clean-up step, index PCR took place. After a further round of size selection with SRISelect, completed libraries were quantified, (Agilent Bioanalyser and qPCR) and diluted for running on an Illumina sequencing instrument (HS4000).

### Processing and Normalization of 10× Data

The results from the sequencing runs were checked manually to confirm that the overall yield and quality were as expected. The data from the instrument were converted to fastq format, the input format required by the 10× software cellranger, and aligned using the human reference GRCh36-1.2.0 available from 10×. The dataset was augmented by integrating counts of a cluster of cholangiocytes from a published dataset (cluster 17 in MacParland S A et al, 10 2018) (17). Cells were annotated as part of different origins, these being primary tissue (PRI), untreated organoids (ORG), and treated organoids (ORGT). Each origin comprises three regions: intrahepatic duct (IHD), common bile duct (CBD), gallbladder (GB). Genes with read counts >0 in at least 3 cells from each batch in at least one origin were maintained for downstream analysis. Low quality cells were removed based on the percentage of UMI mapping to the mitochondrial genome and the number of genes detected by determining outliers (3 median-absolute-deviations) with the routine isOutlier in the package scater (18). Cholangiocytes were isolated by retaining cells expressing at least one of the biliary markers EPCAM, KRT7, KRT19 (with number of counts>3).

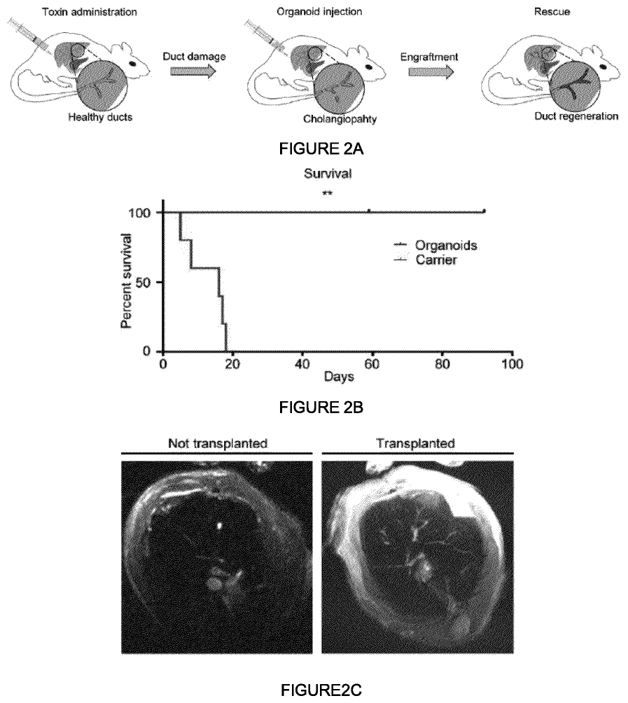

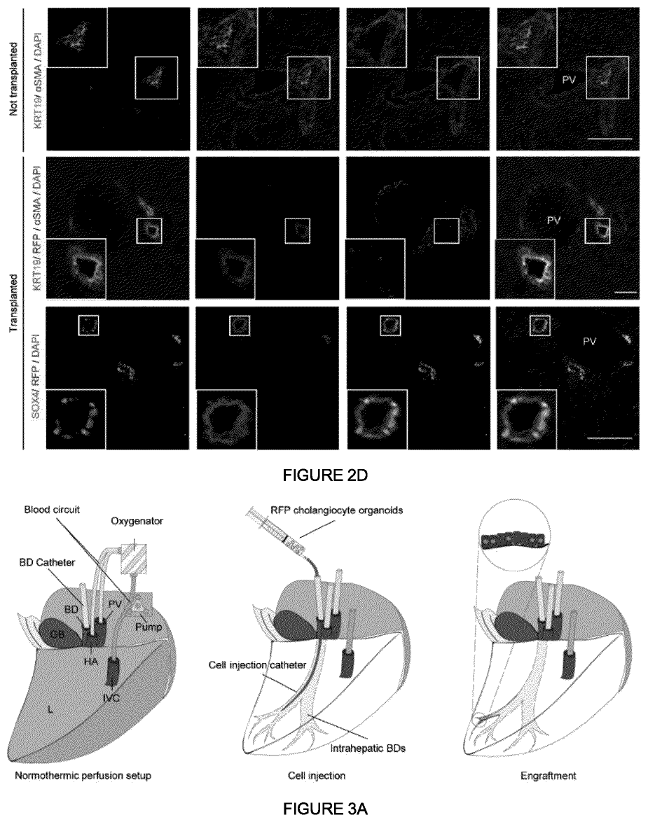

Normalization, identification of highly variable genes and cell cycle regression (regressing out the difference between the G2M and S phase scores) were performed with the Seurat package (19). We employed the routine fastMNN in scran for batch correction (20). Batch corrected samples are shown in FIG. 2A. Small clusters derived by applying the Louvain method for community detection and characterized by cells which were outliers in the percentage of UMI mapping to the mitochondrial genome and the number of genes detected were filtered out.

### Analysis of Normalized 10× Data

The normalized data were clustered using the Louvain method in the Scanpy package (21) by selecting a resolution which generated 3 clusters and with 10 random initialisations. Similarity between Louvain clusters and origin annotations was assessed using the Adjusted Rand Index (ARI) and the Adjusted Mutual Information (AMI). Both measures lie in the interval [0,1], where a value close to 0 indicates random labelling and exactly 1 means that the two partitions are identical. The average value calculated on the different partitions obtained by random initializations was >0.95 for both measures, indicating a high correspondence between origins and clusters. The same analysis performed on regions showed poor matching between regions and clusters, suggesting similarity in the transcriptional profile of cells located in different regions. Transcriptional similarity was quantified at origin and region resolution by estimating the connectivity of data manifold partitions within the partition-based graph abstraction (PAGA) framework. At the origin resolution, this analysis notably highlighted higher transcriptional similarity between treated organoids and primary tissue than between untreated organoids and primary tissue. Interestingly, at the region resolution we identified higher transcriptional similarity between adjacent locations in primary tissues, with intrahepatic duct and gallbladder having the lowest connectivity value. This association between connectivity and anatomical location, together with the similarity of cells located in different regions, suggested a gradual variation in the transcriptional profile of cells in primary tissue that could be represented as a pseudo-spatial dimension. In this view, we analyzed the primary tissue by applying two methods for pseudo-temporal (or pseudo-spatial) ordering: diffusion pseudo-time (22) and Monocle 2 (23). In Monocle 2 differential expression in pseudotime was calculated using the differential GeneTest routine. Both methods confirmed an association between transcriptional similarity and anatomical location, and allowed the representation of regional markers along a pseudo-spatial dimension. Since the majority of cells had a diffusion pseudotime value >0.65 the density plot was shown in the range [0.65, 0.9] to improve visualization and avoid overcrowding. We then analyzed each region individually in organoids (treated and untreated) and primary tissue to identify potential subpopulations of cells. Due to the relatively small sample sizes, we applied the clustering method SC3, whose high accuracy and robustness is derived combining multiple clustering solutions through a consensus approach (24). SC3 allows the user to pre-define the number of clusters. Because of the arbitrariness of this choice we varied the number of clusters between 1 and 10, calculated the stability of clusters across resolutions (SC3 stability index) and built a clustering tree showing how cells move as the clustering resolution is increased (package clustree), (25). No stable sub-trees were formed within each region, indicating absence of stable clusters defining subpopulations of cells. Regional markers and differentially expressed genes were identified by applying the Wilcoxon-Rank-Sum test (p-value<0.01, |log2fold change|>1) in Scanpy. Gene set, gene ontology and pathway enrichment were performed using the packages GSEA (26) and Enrichr (27).

### Data Availability

10× raw data (fastq files) have been deposited in the repository ArrayExpress with the accession number E-MTAB-8495 22

### Organoid Derivation and Culture

A portion of the cells isolated for scRNAseq was cultured and propagated as organoids using our established methodology (11, 12). Cells were cultured under the same conditions irrespective of their region of origin.

### Immunofluorescence, RNA Extraction and Quantitative Real Time PCR

IF, RNA extraction and QPCR were performed as previously described (11, 12, 28, 29). All QPCR data are presented as the median, interquartile range (IQR) and range (minimum to maximum) of four independent lines unless otherwise stated. Values are relative to the housekeeping gene Hydroxymethylbilane Synthase (HMBS). All IF images were acquired using a Zeiss Axiovert 200M inverted microscope or a Zeiss LSM 700 confocal microscope. Imagej 1.48k software (Wayne Rasband, NIHR, USA,http://imagej.nih.gov/ij) was used for image processing. IF images are representative of 3 different experiments.

### GGT Activity

GGT activity was measured in triplicate using the MaxDiscovery™ gamma-Glutamyl Transferase (GGT) Enzymatic Assay Kit (Bioo scientific) based on the manufacturer's instructions. Error bars represent SD.

### Alkaline Phosphatase Staining

Alkaline phosphatase was carried out using the BCIP/NBT Color Development Substrate (5-bromo-4-chloro-3-indolyl-phosphate/nitro blue tetrazolium) (Promega) according to the manufacturer's instructions.

### Flow Cytometry Analyses

Flow cytometry analyses were performed as previously described (11, 12, 28, 29).

### Bile Acid Treatment

Organoids were incubated for 72 hours with 10 μM CDA (Sigma, C9377-5G) in the presence or absence of 10 μM Z-GS (Santa Cruz, sc-204414).

### Animal Experiments

All animal experiments were performed in accordance with UK Home Office regulations (UK Home Office Project License number PPL 70/8702). Immunodeficient NSG mice (NOD.Cg-Prkdcscid II2rgtm1Wjl/SzJ), which lack B, T and NK lymphocytes, were bred in house, and food and water were available ad libitum before and after procedures. Male animals aged 4-8 weeks were used. Animals were assigned randomly to treatment and control groups. Experiments were performed blinded, and where this was not possible (e.g., due to performance of a surgical procedure), data were analysed blinded to the identity of the experimental groups. Littermate animals were used as controls.

### Cell Delivery

Cholangiocytes were delivered into the liver retrogradely through the extrahepatic biliary tree (14). In brief, a fine bore cannula was placed and secured in the gallbladder. To divert the infusion into the liver, the distal common bile duct was occluded with a clamp. The cells were infused through the cannula in the gallbladder in a total volume of 1 μl/g of total body weight, at a maximum speed of 1 μl/second.

### MDA Sdministration

Cholangiopathy was induced through intraperitoneal (IP) administration of 4,4′-methylene dianiline (MDA) on 3 occasions 7, 5, and 3 days prior to cell delivery at a concentration of 50 μg/g of total body weight. An additional dose of MDA was administered directly into the extrahepatic biliary tree prior to cell delivery as described above.

### Blood Sample Collection

Blood was taken using a 23 g needle directly from the inferior vena cava under terminal anesthesia at the time the animals were electively culled and transferred into 1.5 ml Eppendorf tubes for further processing.

### Blood Sample Processing

The blood samples were routinely processed by the University of Cambridge Core biochemical assay laboratory (CBAL). All of the sample analysis was performed on a Siemens Dimension EXL analyzer using reagents and assay protocols supplied by Siemens.

### Tissue Collection

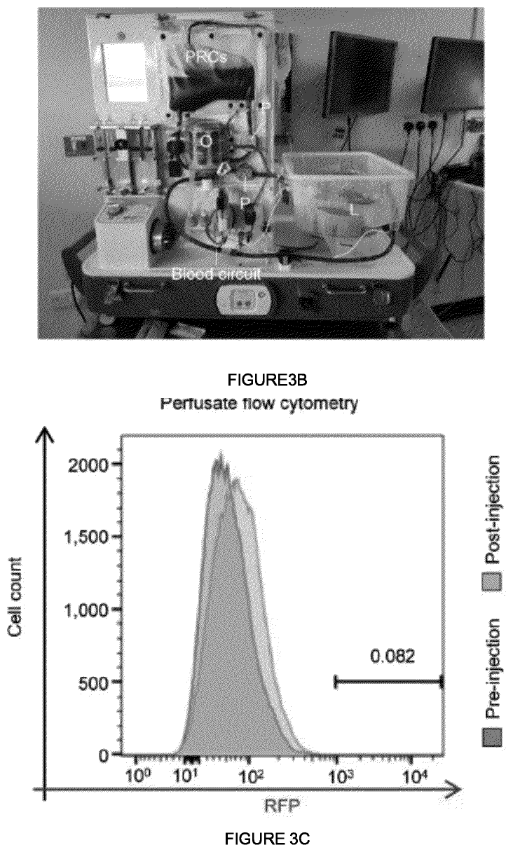

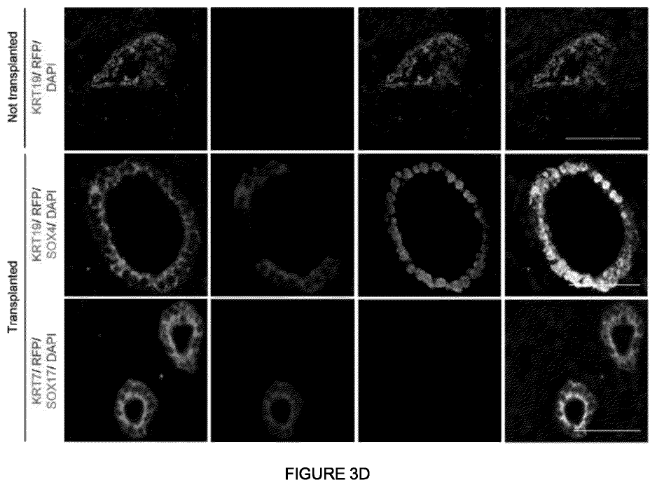

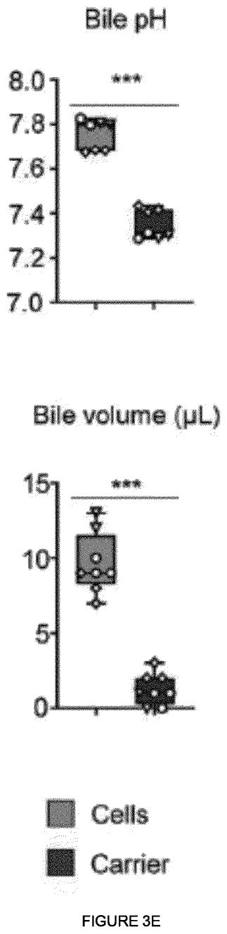

Tissue for sectioning and staining was collected at the end of all animal experiments when the animals were culled, unless otherwise stated. The animals were culled due to due to animal welfare reasons (weight loss, jaundice and clinical deterioration) or electively 3 months after transplantation. Timepoints are indicated on the relevant Kaplan-Meier curves (FIG. 3B).

### Cryosectioning

Excised tissue was fixed in 4% PFA, immersed in sucrose solution overnight, mounted inoptimal cutting temperature (OCT) compound and stored at −80° C. until sectioning. Sections were cut to a thickness of 6-10 μm using a cryostat microtome and mounted on microscopy slides for further analysis.

### Haematoxylin and Eosin (H&E) Staining

H&E staining was performed by the histology service of Addenbrooke's hospital or using Sigma-Aldrich reagents according to the manufacturer's instructions. Briefly, tissue sections were hydrated, treated with Meyer's Haematoxylin solution for 5 minutes (Sigma-Aldrich), washed with warm tap water for 15 minutes, placed in distilled water for 30-60 seconds and treated with eosin solution (Sigma-Aldrich) for 30-60 seconds. The sections were subsequently dehydrated and mounted using the Eukitt@ quick-hardening mounting medium (Sigma-Aldrich).

### Histology

Histology sections were reviewed by an independent histopathologist with a special interest in hepatobiliary histology (SD).

### Quantification of Transplanted Cells in Mouse Liver

For each animal 3 random sections were analyzed, with different lobes being assessed. A total of 49,846 cells were analyzed, approximately 10,000 cells per animal.

### MR Imaging

Magnetic resonance cholangio-pancreatography was performed after sacrifice of the animals. MRCP was performed at 9.4T using a Bruker BioSpec 94/20 system (Bruker, Ettlingen, Germany). For higher signal to noise ratio to give improved visualisation of thebiliary ducts a two-dimensional sequence was used with slightly varied parameters (spaced echoes at 11 ms intervals to give an effective echo time of 110 ms; repetition time 5741 ms; matrix size of 256×256; field of view of 4.33×5.35cm2 yielding a planar resolution of 170×200 μm2). Slices were acquired coronally through the liver and gall bladder with a thickness of 0.6 mm. For this acquisition, a volume coil was used to reduce the impact of radiofrequency inhomogeneity.

To examine the biliary tree, images were prepared by maximum intensity projections. Structural imaging to rule out neoplastic growths was performed using a T1-weighted 3D FLASH (fast low-angle shot) sequence with a flip angle of 25°, repetition time of 14 ms and anecho time of 7 ms. The matrix was 512×256×256 with a field of view of 5.12×2.56×2.56 cm3 for a final isotropic resolution of 100 μm.

Volume rendered images of the biliary tree were generated from source data using Osirix software. The region of interest was segmented from the remaining data manually. The MRCP images were reviewed by 2 independent radiologists with a special interest in hepatobiliary radiology (EMG, SU).

### Ex Vivo Normothermic Perfusion of Donor Livers

The metra (OrganOx, Oxford, UK) normothermic liver perfusion device was used for ex vivo perfusion of human livers as previously described (15, 30). The machine, which is clinically used for preservation of livers for transplantation (15) enables prolonged automated organ preservation by perfusing it with ABO-blood group-compatible normothermic oxygenated blood. The perfusion device incorporates online blood gas measurement, as well as software-controlled algorithms to maintain pH, PO2 and PCO2 (within physiological limits), temperature and mean arterial pressure within physiological normal limits. In brief, the hepatic artery, portal vein, inferior vena cava and bile duct were cannulated, connected to the device and perfusion commenced.

### Bile Duct Cannulation

Cannulation of the bile duct was achieved by inserting two Fr sheaths into the common bile duct under fluoroscopy guidance, followed by cannulation of the left and right hepatic ducts and subsequently segment 3 and segment 5 ducts respectively, using two 2.7 Fr microcatheters via the sheaths. Peripheral placement of the microcatheters was confirmed by cholangiogram with small amount of ionic contrast medium. Cells were injected into segment 3 and carrier was injected into segment 5.

### Cell Delivery

RFP-expressing organoids were mechanically dissociated to a mixture of small clumps and single cells and approximately 10×106 RFP-expressing cells were administered in a peripheral duct of segment 3 with a distribution area of ˜2 cm3, which was cannulated under fluoroscopic guidance to maximize cell delivery (see Bile duct cannulation section)). Carrier medium was delivered in a peripheral branch of segment 5 using the same technique and the organ was maintained on NMP for up to 100 hours.

### Quantification of Transplanted Cells in Human Livers

3 human livers injected with RFP-labelled gallbladder organoids were analysed. Sections were obtained from the area of the distribution of the cells (˜2 cm3). 5 sections per liver and a total of 4,463 cells were analysed.

### Bile Aspiration

Bile duct cannulation was performed as described in the relevant section. Following cannulation, 2 microfluidic catheters (CMA Microdialysis Catheter, Harvard Bioscience Inc, USA) were placed into the respective segmental ducts using a guide wire exchange technique. The inner and outer shaft of the catheter and the inlet and outlet tubing are made of polyurethane and the membrane composed of polyarylethersulphone with a membrane pore size of 100 kDa and outer diameter of 0.4 mm. The inlet tubing for each catheter was connected to a portable battery driven CMA 107 Microdialysis Pump (Harvard Bioscience Inc, USA) and the pump was set to aspirate at a rate of 1 μl/min.

### Bile Volume and pH Measurements

Measurements were performed in n=3 different livers. A minimum of 2 repeat measurements were performed for each liver increasing to 3 where possible, as previously described (27). Bile volume was normalised over the volume of the bile ducts producing it, which corresponds to the volume of distribution of the cells or the carrier in the control arm. This was calculated using the volume of the contrast medium required to delineate these ducts on cholangiogram. Please note all catheters were primed prior to volume measurements.

### Ultrasound Imaging

The liver was imaged ex-vivo in a normothermic perfusion device using a Hitachi Aloka Arrieta V70 and a 10 Mhz hand-held probe. Images were obtained in axial and sagittal planes and assessment of the portal vein, hepatic veins and their major branches was carried out. The intrahepatic bile ducts were also assessed, with particular attention to segment 3 where the organoids had been instilled, and a control area in segment 5 receiving carrier.

Statistical analysis

All statistical analyses were performed using GraphPad Prism 6. For small sample sizes where descriptive statistics are not appropriate, individual data points were plotted. For comparison between mean values a 2-sided Student's t-test was used to calculate statistical significance. The normal distribution of our values was confirmed using the D'Agostino & Pearson omnibus normality test where appropriate. Variance between samples was tested using the Brown-Forsythe test. For comparing multiple groups to a reference group one-way ANOVA followed by Dunnett's test was used between groups with equal variance, while the Kruskal-Wallis test followed by Dunn's test was applied for groups with unequal variance. Survival was compared using log-rank (Mantel-Cox) tests. Where the number of replicates (n) is given this refers to organoid lines or number of different animals unless otherwise stated.

For animal experiments, group sizes were estimated based on previous study variance. Final animal group sizes were chosen to allow elective culling at different time point while maintaining n>4 animals surviving past 30 days to ensure reproducibility. No statistical methods were used to calculate sample size. No formal randomization method was used to assign animals to study groups. However, littermate animals from a cage were randomly assigned to experimental or control groups by a technician not involved in the study. No animals were excluded from the analysis. Blinding was used for radiology imaging.

### 2. Results

We generated a single-cell map of the human biliary tree to use as a framework to characterise cholangiocyte organoids. To this end, a fraction of the primary cholangiocytes isolated for scRNAseq from each region (IHD, CDB, GB) were propagated as organoids using our established conditions (3, 16). The resulting organoids expressed cholangiocyte markers (KRT7, KRT19, SOX9, HNF1B, CFTR) displayed comparable functionality (ALP, GGT activity) and similar expansion potential regardless of their region of origin. To further explore these similarities, we performed scRNAseq on these organoids (2 lines per region; GB: 5859 cells; CBD 5321 cells; IHD 6641 cells). UMAP and PCA analyses demonstrated that organoids exhibited overlapping transcriptomic profiles (FIG. 1A) indicating that cholangiocytes grown in vitro assume a similar transcriptional signature independent of their region of origin. Of note, regressing cell cycle-related genes did not change these observations excluding that a common “proliferation” signature could mask differences between organoids of different spatial origins. Furthermore, we did not detect any cells co-expressing known somatic stem cell markers (LGR5, PROM1, TACSTD2, NCAM), excluding the possibility that organoid similarities reflect a common progenitor/stem cell identity.

We then compared organoids from different regions with primary cholangiocytes to explore if these similarities corresponded to loss of their original regional identity in vitro (FIG. 1A).

Organoids and primary cells following cell cycle regression shared a core transcriptional profile reflecting their common cholangiocyte nature, which was illustrated by their proximity in UMAP space and high PAGA connectivity when compared to different liver cell types, such as stellate cells and LSECs. However, DEG analyses highlighted downregulation of region-specific markers, such as SLC13A1 and SLC26A3 (FIG. 2B); while Gene Ontology (GO) and Gene Set Enrichment Analyses (GSEA) identified these DEGs as factors facilitating the adaptation of cholangiocytes to their respective microenvironments, e.g. bile acid vs. culture medium processing genes. Furthermore, we confirmed upregulation of YAP target genes in organoids, in accordance with previous reports (14). Consequently, primary cholangiocytes propagated as organoids adapt to their new microenvironment by maintaining their core transcriptional signature, while losing the expression of markers specific to their region of origin.

To explore the mechanisms controlling cholangiocyte identity, we decided to add bile in our culture conditions as the principal determinant of the cholangiocyte microenvironment.

Different organoids (IHD, CBD, GB) were treated with human gallbladder bile for 72 hours and then characterized using scRNAseq (FIG. 1A,) (GB: 3815 cells; CBD 3224 cells; IHD 3653 cells). UMAP and PCA revealed that treated organoids assumed a new overlapping gene expression profile (FIG. 2A confirming a shared capacity to adapt to exposure to bile. Importantly, PAGA and DEG analyses showed that this transcriptional profile was shifted towards a gallbladder identity (FIG. 1B). To characterise the factors controlling this transition, we interrogated differentially expressed genes in bile-treated organoids. GO, GSEA and UMAP analyses confirmed the induction of region-specific markers (SOX17, MUC13, FGF19; FIG. 2B) and revealed upregulation of bile acid receptor pathways and downstream targets (NR1H4/FXR, NR112, NROB2, SLC51A, FGF19, ABCA1, PPARG; FIG. 1B). Of note, these results were validated through activation and inhibition of the Farnesoid X receptor (FXR), using chenodeoxycholic acid and z-22 guggulsterone respectively (FIG. 1C-1D), thereby confirming that regardless of their origin, cholangiocytes grown in vitro can respond and adapt to environmental stimuli. Together, these results suggest that cholangiocyte organoids could assume different regional identities when instructed by the appropriate niche factors.

To validate cholangiocyte plasticity and explore its functional implications, we decided to assess if organoids from one region of the biliary tree could repair a different region following transplantation. For this, we induced cholangiopathy in immunodeficient mice using 4,4′-methylenedianiline (MDA) (17) (FIG. 2A-2B) and attempted to rescue the phenotype with intraductal delivery (18) of human gallbladder organoids expressing Red Fluorescent Protein-expressing (RFP) that were produced without an FXR agonist. Control animals receiving carrier medium without cells lost weight and died within 3 weeks (FIG. 2B), developing cholestasis and cholangiopathy demonstrated by IF, histology and Magnetic Resonance Cholangio-Pancreatography (MRCP) (FIG. 2C). On the contrary, animals receiving organoids were electively culled at the end of the experiment and survived for up to 3 months with resolution of cholangiopathy and normal serum biochemistry (FIG. 2B-2C). The transplanted gallbladder cholangiocytes engrafted in various size intrahepatic ducts (FIG. 2D) corresponding to ˜25-55% of the regenerated biliary epithelium.

Core biliary markers (KRT7, KRT19, CFTR) were also expressed, while we observed YAP activation both in engrafted and native cells in accordance with previous reports (13). Of note, we never observed expression of other hepatic lineage markers such as albumin indicating that cholangiocyte organoid plasticity is likely to be limited to their biliary lineage. Furthermore, the engrafted cells expressed proliferation markers at similar levels to native mouse cholangiocytes; while abnormal growth or tumour formation was never noticed in all the analyses performed (FIG. 2C, 2D), including T1 weighed body MR imaging at the end of the experiment. Thus, organoid transplantation provides the healthy cells required to repair the damaged epithelium and rescue acute injury.

Finally, to ensure that our results are not specific to the intrahepatic compartment or gallbladder organoids, we used our established methodology (3) to transplant common bile duct-derived cholangiocyte organoids derived without an FXR agonist in the gallbladder of immunocompromised mice. The engrafted cells exhibited loss of common bile duct makers and upregulation of gallbladder markers, confirming that our previous findings apply to different compartments of the biliary tree and to organoids of different origin. Taken together, these results establish that cholangiocytes from different regions of the biliary tree are interchangeable and suggest that extrahepatic cells can be used to repair acute intrahepatic duct injury.

Cell transplantation experiments in mouse models are extremely useful but are not always predictive of therapeutic outcome (19). Furthermore, the mouse liver microenvironment is different to human, raising the possibility that our results may not translate between species. To address these challenges, we developed a new model for cell-based therapy in human utilizing ex vivo organ perfusion (20). Ex-vivo Normothermic Perfusion (NMP) was developed to improve organ preservation and reduce ischaemia-reperfusion injury by circulating warm oxygenated blood through liver grafts prior to transplantation. Importantly, the biliary tree is particularly susceptible to ischaemia which results in duct damage (21, 22). Low bile pH (<12 7.5) during NMP is used as a predictor of this type of cholangiopathy (23).

To assess the therapeutic potential of our cells for repairing human bile ducts, RFP gallbladder organoids derived without an FXR agonist were injected in the intrahepatic ducts of deceased transplant donor livers (n=3) with a bile pH<7.5 at the start of the experiment, signifying ischaemic duct injury. The organs were perfused with oxygenated blood and nutrients at normal body temperature (20); FIG. 3A-43B) for up to 100 hours in order to maintain a near-physiological microenvironment. Importantly, the organoids were delivered in a terminal branch of the intrahepatic ducts under fluoroscopic guidance to minimize the area of distribution of the cells and maximize cell density. At the end of the experiment, ultrasound imaging revealed no evidence of duct dilatation or obstruction, while RFP-expressing cells were not detected in the perfusate by flow cytometry, confirming that the injected cells remained in the biliary compartment (FIG. 3C). More importantly, the transplanted organoids engrafted in the intrahepatic biliary tree (FIG. 3D), with RFP cells regenerating ˜40-85% of the injected ducts; and expressing key biliary markers (KRT7, KRT19, CFTR, GGT). Thus, at the end of the experiment, the injected ducts consisted of a mixture of native and transplanted cholangiocytes, with multiple transition points between donor and recipient cells and no evidence of cholangiopathy (FIG. 3D).

Conversely, control ducts not receiving cells demonstrated evidence of ischaemic injury with loss of epithelial continuity and sloughing of cells in the duct lumen (FIG. 3D). We subsequently characterised the impact of engraftment on organ function. Physiologically, cholangiocytes modify the composition and pH of bile through water transfer and bicarbonate secretion (6). Therefore, we compared the bile from organoid-injected vs. carrier-injected ducts. Accordingly, bile aspirated from ducts injected with cells exhibited higher pH and volume (FIG. 3E) confirming that transplanted cholangiocytes retain their function to modify bile composition. Together, these results provide the first proof-of-principle that perfused organs can be used to ascertain functional engraftment of human cells and validate our mouse data by showing that cholangiocytes are interchangeable for transplantation in human organs.

Our results show that the biliary epithelium is composed of cholangiocytes with diverse transcriptional profiles which are determined by their local environment. This diversity is lost in organoid culture due to the lack of niche stimuli. However, organoids can adapt appropriately to local environmental cues both in vitro and following transplantation, restore the expression of region-specific markers and assume different regional identities. Thus, organoids from a single region could potentially repair the entirety of the biliary tree. This plasticity could have significant implications for regenerative medicine. Indeed, although autologous cell-based therapy potentially avoids the need for immunosuppression its application for primary organoids is limited by the impact of disease on the epithelium. However, cholangiopathies belong to a family of localising diseases, affecting predominantly specific regions of an organ (24).

Consequently, our results provide proof-of-concept that cholangiocytes from spared regions, such as the gallbladder, could be used for autologous cell-based therapy to repair human intrahepatic bile ducts, which constitute the most common site of injury in cholangiopathies. Moreover, our novel model for cell engraftment in human perfused organs paves the road for the use of ex vivo cell-based therapy to improve graft function prior to transplantation, which could ultimately increase the number of useable organs and reduce pressure on the transplant waiting list. In this context, quality controlled and readily available allogeneic cholangiocyte organoids from a cell bank could be used routinely in the future to prevent ischaemic cholangiopathy in organs at risk of biliary injury (e.g. low bile pH), since the organ recipients will receive immunosuppression as part of their standard care. Importantly, our results provide proof-of-principle for the transplantation of organoids in human organs which could expedite regulatory approval and fast-tack first-in-man trials. Ultimately, the same approach could also be applied to a variety of ex vivo perfused organs and cell types to validate functional cell engraftment, demonstrate safety, improve cell transplantation technique and efficacy and accelerate clinical translation of new cell-based therapies.

### REFERENCES

1. J. Drost, H. Clevers, Dev. 144, 968-975 (2017).
2. R. H. Squires, et al Hepatology. 60, 362-98 (2014).
3. F. Sampaziotis, et al. Nat. Med. 23, 954-963 (2017).
4. K. Parikh, et al Nature. 567, 49-55 (2019).
5. C. A. Rimland et al. Hepatology (2020), 3 doi: 10.1002/hep.31252.
6. J. H. Tabibian et al. Compr. Physiol. 3, 541-565 (2013).
7. A. I. Masyuk, et al Hepatology. 43, 7 S75-S81 (2006).
8. K.-S. Yoo, et al Gut Liver. 10, 851-8 (2016).
9. S. J. L. B. Zweers et al Hepatology. 55, 575-583 (2012).
10. Y. Zong et al Int. J. Biochem. Cell Biol. 43, 257-64 (2011).
11. K. Si-Tayeb, et al Dev. Cell. 18, 175-189 (2010).
12. N. Aizarani et al Nature. 572, 199-204 (2019).
13. B. J. Pepe-Mooney, et al Cell Stem Cell. 25, 23-38.e8 (2019).
14. L. Planas-Paz, et al Cell Stem Cell. 25, 39-53.e10 (2019).
15. A. Lanzini, BILE. Encycl. Food Sci. Nutr., 471-478 (2003).
16. O. C. Tysoe, et al Nat. Protoc. 14, 1884-1925 (2019)
17. M.-O. Lee et al Toxicol. Pathol. 36, 660-673 (2008).
18. N. L. Berntsen et al Am. J. Physiol.-Gastrointest. Liver Physiol. 314, G349-G359 (2018).
19. C. Arber et al Semin. Hematol. 50, 131-144 (2013)
20. D. Nasralla et al Nature. 557, 6 50-56 (2018).
21. A. I. Skaro et al Surgery. 146, 543-553 (2009).
22. C. K. Enestvedt et al Liver Transpl. 19, 965-72 (2013).
23. C. J. E. Watson et al Am. J. Transplant. 18, 2005-2020 (2018).
24. R. G. Farmer et al Gastroenterology. 68, 627-35 (1975).
25. S. A. MacParland et al Nat. Commun. 9, 4383 (2018).
26. D. J. McCarthy et al Bioinformatics. 33, 1179-1186 (2017).
27. A. Butler, et al Nat. Biotechnol. 36, 411-420 (2018).
28. L. Haghverdi et al Nat. Biotechnol. 36, 421-427 (2018).
29. F. A. Wolf, et al Genome Biol. 19 (2018), doi: 10.1186/s13059-017-1382-0.
30. L. Haghverdi, et al Nat. Methods. 13, 845-848 (2016).
31. X. Qiu, Q. et al Nat. Methods. 14, 979-19 982 (2017).
32. V. Y. Kiselev, et al Nat. Methods. 14, 483-486 (2017).
33. L. Zappia, A. et al Gigascience. 7 (2018), doi: 10.1093/gigascience/giy083.
34. A. Subramanian, et al. Proc. Natl. Acad. Sci. U. S. A. 102, 15545-15550 (2005).
35. M. V. Kuleshov et al. Nucleic Acids Res. 44, W90-W97 (2016).
36. F. Sampaziotis et al. Nat. Biotechnol. 33, 845-852 17 (2015).
37. F. Sampaziotis, et al. Nat. Protoc. 12, 814-827 (2017).
38. R. Ravikumar, et al. Am. J. Transplant. 16, 1779-1787 (2016)

1. A method for expanding cholangiocytes in vitro comprising:
(i) providing a population of cholangiocytes and;
(ii) culturing the population in the presence of a farnesoid X receptor (FXR) agonist to produce an expanded population of FXR-treated cholangiocytes.

2. The method according to claim 1 wherein the farnesoid X receptor (FXR) agonist is chenodeoxylic acid (CDA) or obeticholic acid (OCA)

3. The method according to claim 1 wherein the cholangiocytes are primary cholangiocytes or cholangiocytes differentiated from iPSCs.

4. The method according to claim 1 wherein the population is cultured in an expansion medium comprising the farnesoid X receptor (FXR) agonist, an epidermal growth factor (EGF), a canonical Wnt signalling inhibitor and a non-canonical Wnt signalling potentiator, to produce the expanded population of FXR-treated cholangiocytes.

5. The method according to claim 4 wherein the FXR-treated cholangiocytes form organoids in the expansion medium.

6. The method according to claim 1 wherein the cholangiocytes are extrahepatic cholangiocytes, intrahepatic cholangiocytes, or gallbladder cholangiocytes.

7. (canceled)

8. (canceled)

9. (canceled)

10. The method according to claim 4 wherein the non-canonical Wnt signalling potentiator is a Wnt agonist.

11. The method according to claim 10 wherein the Wnt agonist is R-spondin.

12. The method according to claim 4 wherein the canonical Wnt signalling inhibitor is Dickkopf-related protein 1 (DKK-1).

13. (canceled)

14. The method according to claim 4 wherein population is cultured in 3D culture in the expansion medium.

15. (canceled)

16. The method according to claim 4 wherein expansion medium comprises a scaffold matrix and a nutrient medium supplemented with (i) EGF, (ii) the canonical Wnt inhibitor (iii) the non-canonical Wnt potentiator and (iv) the FXR agonist.

17. (canceled)

18. (canceled)

19. (canceled)

20. (canceled)

21. (canceled)

22. (canceled)

23. (canceled)

24. (canceled)

25. The method according to claim 4 wherein the cholangiocytes are cultured in the expansion medium for 20 or more passages.

26. The method according to claim 5 wherein the method comprises disrupting cholangiocyte organoids such that the expanded population comprises individual cells.

27. The method according to claim 1 comprising seeding the expanded population of FXR-treated cholangiocytes into a biocompatible scaffold, optionally within the scaffold matrix is a decellularised human or non-human tissue extracellular matrix.

28. The method according to claim 27 comprising culturing the biocompatible scaffold in an expansion medium comprising an RXR agonist, epidermal growth factor (EGF), a canonical Wnt signalling inhibitor and a non-canonical Wnt/signalling potentiator, such that the cholangiocytes populate the scaffold.

29. (canceled)

30. (canceled)

31. (canceled)

32. An isolated population of FXR-treated cholangiocytes produced by the method of claim 1.

33. (canceled)

34. The population according to claim 32 wherein the FXR treated cholangiocytes are within a biocompatible scaffold, optionally wherein the decellularised scaffold matrix is a human or non-human tissue extracellular matrix.

35. (canceled)

36. (canceled)

37. (canceled)

38. A biocompatible scaffold comprising the isolated population according to claim 32.

39. (canceled)

40. A method of treating a patient with a biliary disorder comprising;
administering the population of isolated FXR-treated cholangiocytes according to claim 32 to an individual in need thereof.

41. The method according to claim 40 further comprising administering an FXR agonist to the individual.

42. (canceled)

43. (canceled)

44. (canceled)

45. (canceled)

46. (canceled)

47. A method of screening a compound comprising;
contacting the population of isolated FXR-treated cholangiocytes according to claim 32 with a test compound, and;
determining the effect of the test compound on said the FXR treated cholangiocytes or scaffold and/or the effect of said FXR treated cholangiocytes or scaffold on the test compound.

48. (canceled)

49. (canceled)

50. (canceled)

51. (canceled)
